(19) United States
(12) Patent Application Publication
(10) Pub. No.: US 2011/0281949 A1
(43) Pub. Date: Nov. 17, 2011

(54) METHODS AND COMPOSITIONS FOR TREATING NEURONAL DAMAGE AND MODULATING TRANSIENT RECEPTOR POTENTIAL CHANNELS

(76) Inventors: Baruch Minke, Jerusalem (IL); Moshe Parnas, Mevaseret Tziyon (IL)

(21) Appl. No.: 13/020,117

(22) Filed: Feb. 3, 2011

Related U.S. Application Data
(63) Continuation-in-part of application No. PCT/IB2009/053292, filed on Jul. 29, 2009.
(60) Provisional application No. 61/085,920, filed on Aug. 4, 2008.

(51) Int. Cl. A61K 31/192; C07C 35/12; C07C 35/18; C07C 47/232; C07C 59/52; C07C 35/30; C07C 49/437; A61K 31/05; A61K 31/045; A61K 31/11; A61K 31/125; A61P 17/04; A61P 35/00; A61P 17/00; A61P 25/00; C07C 39/06

(52) U.S. Cl. 514/570; 568/781; 568/829; 568/825; 568/425; 562/478; 568/820; 568/374; 514/731; 514/729; 514/701; 514/692

(57) ABSTRACT

Use of alkyl phenol and monocyclic monoterpene-derived compounds in the treatment of neuronal damage and in inhibiting activity of TRPC and non-thermo-TRPM channels, and use of bicyclic monoterpene-derived compounds in activating TRPC and non-thermo-TRPM channels.

### FIELD OF THE INVENTION

The present invention concerns use of alkyl phenol and monocyclic monoterpene-derived compounds, particularly in the treatment of neuronal damage and inhibiting activity of TRPC and non-thermo-TRPM channels. The present invention also concerns the use of bicyclic monoterpene-derived compounds in activating TRPC and non-thermo-TRPM channels.

### BACKGROUND OF THE INVENTION

### TRP Channels

Transient Receptor Potential (TRP) channels are essential components of biological sensors that detect changes in their surroundings in response to various stimuli such as cold or hot temperatures, natural chemical compounds and mechanical stimuli. TRP channels are Ca²⁺-permeable and are involved in photoreception, pheromone sensing, taste perception, thermo-sensation, pain perception, mechano-sensation, perception of pungent compounds, renal Ca²⁺/Mg²⁺ maintenance, smooth muscle tone, blood pressure regulation, and the like. TRP channels form an evolutionary conserved cation channel family generally considered as consisting of 7 subfamilies that include nearly 30 human members (Minke B et al, 2002). The founding member of this family, TRP, was discovered in Drosophila (Minke B et al, 1975). The seven TRP subfamilies are designated:

1. TRPC (Canonical or classical)

2. TRPM (Melastatin)

3. TRPN (NompC)

4. TRPV (Vanilloid receptor)

5. TRPA (ANKTM1)

6. TRPP (Polycystin), and

7. TRPML (mucolipin).

In addition to the above grouping based on sequence homology, TRP channels are divided functionally into those activated by hot or cold temperatures, designated thermo-TRPs, and the non-thermo TRPs. Thermo-TRPs include TRPV1-4, TRPM8 and TRPA1, while non-thermo TRPs include several other TRPM family members and the TRPC subfamily. These channels are involved in thermal sensation and are also believed to be regulated by pungent natural compounds that elicit hot or cold sensations, such as Compound IX, Compound X, Compound XI and Compound XII (Macpherson L J et al, 2006; Xu H et al, 2006), although Compound IX has been reported as having only a minimal effect on TRPM8 (Vogt-Eisele AK, 2007).

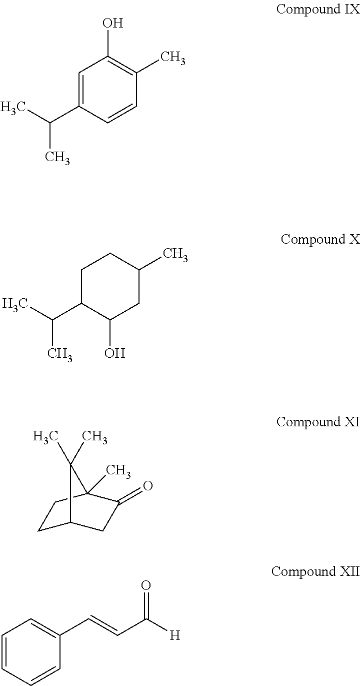

A major difficulty in the study of TRP channels has been the lack of available pharmacological agents that activate or inhibit many members of the classes of TRP subfamilies, particularly the non-thermo TRPs, such as the TRPC and non-thermo-TRPM subfamilies. The prior research does not appear to have addressed whether compounds such as those described above modulate non-thermo-TRP channels. In fact, these compounds were previously believed to have activity specifically for thermo-TRPs, because of their effects on thermal sensation (Macpherson L J et al, 2006).

2-Aminoethoxydiphenyl borate (2-APB) apparently inhibits TRPL channels (Chorna-Ornan I et al, 2001) and activates TRPV3 (Hu et al, 2004). Compound IX (Xu et al, 2006) and Compound XI and Compound XVII (Moqrich A, 2005) are believed to activate TRPV3; however, none of these references disclose or suggest an inhibitory affect of any of these compounds on non-thermo-TRP such as TRPL channels.

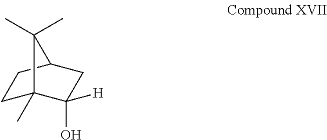

WO2009/004071 to Armin Schneider et al (Sygnis Bioscience), titled “Use of Piperine and Derivatives Thereof for the Therapy of Neurological Conditions” suggests use of agonists of TRPV1, a thermo-TRP, having the formula

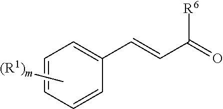

wherein R¹ represents an alkoxy, cycloalkyl, or halogen moiety, for treating a neuronal condition. The compounds described therein function by activation of thermo-TRP channels, as opposed to compounds of the present invention, which inhibit non-thermo-TRP channels.

WO2006/038070 to Kazimierz Babinski et al (Painreceptor Pharma Corp.) titled “Compositions and Methods for Modulating Gated Ion Channels” suggests use of TRPV1 agonist compounds having the general formula:

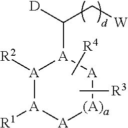

where A, R¹-R⁴, D, and W may be a wide variety of substituents. These compounds also function by activation of thermo-TRP channels.

WO2008/065666 to Arik Moussaieff et al (Yissum Research and Development Co.) titled “Uses of Incensole, Incensole Acetate and Derivatives Thereof” describes use of TRPV3 agonists having the formula

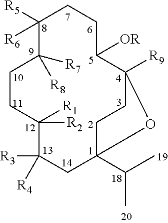

for treatment of inflammatory-associated conditions and mood disorders, and also suggests the use of other TRPV3 agonists, such as Compound IX, in the treatment of mood disorders. The publication further suggests that compounds of the above formula (but not TRPV3 agonists in general) may be useful for conditions resulting from injury, trauma, or CNS neurodegenerative diseases.

An article by Aleksic (Med. Klin. 56:1751-2, 1961) suggests that moxisylyte (thymoxamine) has an anesthetic effect on patients suffering from post-traumatic headaches.

### The Role of TRPC Subfamily Members in Acute Brain Injury

Death of CNS neurons during acute brain injury occurs from a combination of factors, including hypoxia. However, it is known that a constitutive ATP-dependent process is required to keep members of the TRPC subfamily closed, even when no external stimulus is applied. Upon hypoxia these channels open spontaneously, leading to toxic increases in intracellular Ca²⁺ and cell death (Agam K et al). Similarly, non-thermo-TRPM channels such as TRPM7 are activated by oxidative stress and oxygen free radicals downstream of excitotoxic signal pathways, thus mediating anoxic neuronal death. siRNA reduction of expression of these channels protects from neuronal death by ischemia (Aarts M et al, 2003 and Aarts M et al, 2005). Thus, inhibition of these channels constitutes an effective strategy for preventing death of CNS neurons following ischemia and acute brain injury (Aarts M et al, 2003).

Currently, there is no effective treatment for treating neuronal damage, particularly acute neuronal damage, and embodiments of the present invention address this need.

### SUMMARY OF THE INVENTION

Embodiments of the present invention are directed to the use of alkyl phenol and monocyclic monoterpene-derived compounds in the treatment of neuronal damage and in inhibiting activity of TRPC and non-thermo-TRPM channels. Embodiments of the invention are also directed to use of bicyclic monoterpene-derived compounds in activating TRPC and non-thermo-TRPM channels.

In another embodiment, the present invention provides a composition comprising one or more compounds of Formula I:

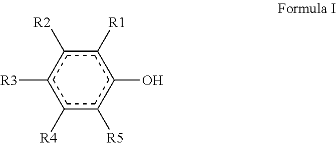

or a hydrous or anhydrous salt or metastable or stable polymorph thereof,
for treating or inhibiting neuronal damage,
wherein the structure

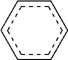

refers to a 6-carbon ring that is either aromatic or has 0, 1, or 2 double bonds,
and wherein R¹, R², R³, R⁴, and R⁵ are as defined herein,
   with the proviso that at least one of R¹, R², R³, R⁴, and R⁵ is C₁₋₄ alkyl that is unsubstituted or substituted as recited above.

In another embodiment, the compound utilized has the structure of Formula II:

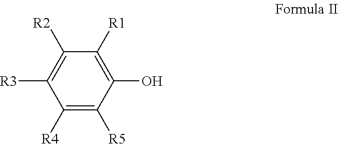

wherein R¹, R², R³, R⁴, and R⁵ are as defined herein.

In another embodiment, the compound has the structure of Formula III:

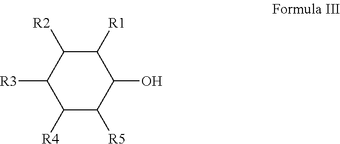

wherein R¹, R², R³, R⁴, and R⁵ are as defined herein.

In other embodiments, the present invention provides a composition including one or more compounds of Formula VIII:

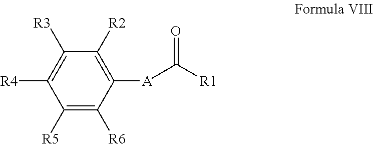

or a hydrous or anhydrous salt or metastable or stable polymorph thereof,
for treating or inhibiting neuronal damage,
wherein R¹ is selected from the group consisting of H and —OH and A, R², R³, R⁴, R⁵, and R⁶ are as defined herein.

In other embodiments, the present invention provides a composition comprising a compound of Formula IV:

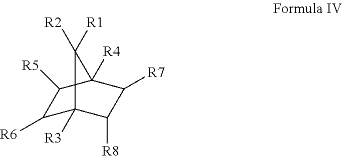

or a hydrous or anhydrous salt or metastable or stable polymorph thereof,
wherein R¹—R⁷ are as defined herein,
for activating a TRP channel selected from the group consisting of a TRPC channel and a non-thermo-TRPM channel.

As provided herein, it was decided to test compounds known to activate TRPV3 (Vogt-Eisele A K et al, 2007), in particular (a) OH-substituted aromatic monocyclic monoterpenoid compounds such as Compound IX, Compound XIII, and Compound XIV and (b) OH-substituted non-aromatic monocyclic monoterpenoid compounds such as Compound X and Compound XVI inhibit TRPC-family channels and non-thermo-TRPM channels. Interestingly, despite their agonist activity on TRPV3 and other thermo-TRPM channels, these compounds exhibited antagonist activity against the tested channels (FIGS. 2-4 and 7-9). Since TRPC-family channels and non-thermo-TRPM channels are highly expressed in mammalian brain (FIG. 1), it was further decided to test whether the compounds tested herein would have therapeutic activity against brain damage; this was also found to be the case (FIG. 10). These monocyclic natural compounds and derivatives thereof are metabolized in vivo and are not excessively hydrophobic; thus, they are considerably safer than di-aryl compounds such as 2-APB.

Another surprising finding was found when Compound XII and related compounds were tested for antagonist activity against TRPC-family channels and non-thermo-TRPM channels. These compounds were also shown to inhibit these channels (FIG. 5).

Another surprising finding was found when —OH— or ═O-substituted bicyclic monoterpenoid compounds such as Compound XVII and Compound XI were tested for antagonist activity against TRPC-family channels and non-thermo-TRPM channels. These compounds were shown instead to activate these channels (FIG. 6).

As used herein, the prefix “halo-” and the term “halogen” refer to substitution with any of I, Br, Cl, and F.

As used herein, the symbol “═O” includes both aldehyde & ketone compounds.

As used herein, the term “C₁-C₄ alkyl” refers to a non-cyclic aliphatic hydrocarbon of 1 to 4 carbon atoms that is either saturated or unsaturated and is either linear or branched. In case the alkyl includes 2-4 carbon atoms, the alkyl may have 1 or more carbon-carbon double bonds (termed also alkenyl). Examples of alkenyl groups include, without limitation, ethenyl, n-propenyl, and isopropenyl.

Recital of a numerical range e.g. “1-4” herein indicates that the group may contain 1 carbon atom, 2 carbon atoms, 3 carbon atoms, etc., up to and including 4 carbon atoms. The C₁-C₄ alkyl group may be for example methyl, ethyl, propyl, isopropyl, butyl, isobutyl, tertiary butyl, or sec-butyl,

The term “alkoxy,” as used herein, refers to an —O-alkyl group, wherein alkyl is as defined hereinabove.

The term “thioalkoxy,” as used herein, refers to an —S-alkyl group, also referred to as an alkylhio or an alkylsulfanyl group.

The term “haloalkyl,” as used herein, refers to an alkyl moiety as defined hereinabove, wherein 1 or more hydrogen substituents is replaced with a halogen substituent.

The term “haloalkoxy,” as used herein, refers to an —O-haloalkyl group, wherein haloalkyl is as defined hereinabove.

The term “heteroalkyl,” as used herein, refers to an alkyl moiety as defined hereinabove, but wherein 1 or more carbon atoms is replaced with a different atom as recited.

Reference to a particular chemical structure herein includes both racemic mixtures and optically-active forms of the compound. As provided herein, both enantiomers of compounds of the present invention are shown to have therapeutic utility. Thus for brevity, a non-qualified structure may be understood, in various embodiments, as referring to the (+) enantiomer, to the (−) enantiomer, or to mixtures thereof, in various proportions, including but not limited to the racemic mixture. Each specific alternative may be considered as a separate embodiment.

The term “treatment” as used herein, does not refer to complete curing of the disease, as it does not change the mutated genetics causing the disease. This term refers to at least one of: alleviating at least one of the undesired symptoms associated with the disease; improving the quality of life of the subject; decreasing disease-caused mortality, and/or preventing the full manifestation of the disorder before it occurs.

Features shown with some specific embodiments may be incorporated with other embodiments. Thus the scope of the present invention is defined by the appended claims and includes both combinations and sub combinations of the various features described hereinabove as well as variations and modifications thereof, which would occur to persons skilled in the art upon reading the foregoing description.

In the claims, the word “comprise”, and variations thereof such as “comprises”, “comprising” and the like indicate that the components listed are included, but not generally to the exclusion of other components.

### BRIEF DESCRIPTION OF THE FIGURES

FIG. 1. In situ hybridization showing localization of mouse TRPM7 (left panel) and TRPC6 (right panel) channels in sections of mouse brain. Arrow shows the hippocampus, which heavily expresses TRPM7;

FIG. 2: Compound IX inhibits TRPL channels expressed in S2 cells; A. Top panel: Representative I-V curves measured from S2 cells by whole cell patch clamp recordings using voltage ramps from −150 mV to 150 mV within 1 s. Compound IX caused a large decrease in the TRPL-dependent current; Bottom panel: Time course of TRPL current measured from the I-V curves (top) at −90 mV and at 90 mV as indicated, before and after application of Compound IX (500 μM) (n=15); B. Top and bottom panels are as described for A. This is a control experiment showing no effect of Compound IX when the TRPL channels were not expressed (n=7); −90 mV and 90 mV datapoints are essentially superimposed in bottom panel; C. Blocking efficiency of Compound IX as a function of concentration (error bars are SEM, n≧5). Lines over the figure in bottom panels of A-B and in FIGS. 3-6 indicate time of adding compound. “I/I Max” in this and subsequent figures refers to the normalized inhibitory effect relative to the maximal current obtained in each cell. The paradigm from this figure was repeated for FIGS. 3-6;

FIG. 3: Compound XIII and Compound XIV inhibit TRPL channels expressed in S2 cells. Compound XIII (3 mM, A, n=6) and Compound XIV (1 mM, B, n=7) caused a large decrease in the TRPL-dependent current; Top panels in A-B: Representative I-V curves of TRPL-mediated current measured before and after application of the compounds. Bottom panels in A-B: Time course of TRPL-mediated current measured from the I-V curves (top) at −90 mV and at 90 mV as indicated, before and after application of the compounds;

FIG. 4: Compound XII but not Compound XV inhibits TRPL channels expressed in S2 cells. A, C. Compound XII (2 mM, A, n=5) but not Compound XV (5 mM, C, n=7) decreased the TRPL-mediated current. Top and bottom panels are as described for FIG. 3. B. Measurements of blocking efficiency of Compound XII as a function of concentration (error bars are SEM, n≧5).

FIG. 5: Compound X and Compound XVI inhibit TRPL channels expressed in S2 cells. A, C. Compound X (5 mM, A, n=5) and Compound XVI (5 mM, C, n=5) caused a large decrease in the TRPL-dependent current. Top and bottom panels are as described for FIG. 3. B, D. Measurements of blocking efficiency of Compound X (B) and Compound XVI (D) as a function of concentration (error bars are SEM, n≧5).

FIG. 6: Compound XVII activates TRPL channels expressed in S2 cells. A-B. Compound XVII (5 mM, n≧5) activated the TRPL-mediated current when channels were almost closed (A) and when spontaneous activity of the TRPL channel was observed (B). Top and bottom panels are as described for FIG. 3. Similar results were obtained when applying Compound XI instead of Compound XVII. C. Control experiment showing no effect of Compound XVII when TRPL channels were not expressed; −90 mV and 90 mV datapoints are essentially superimposed in bottom panel (n=5).

FIG. 7: Compound IX inhibits the native TRPL channels in photoreceptor cells. A. Representative whole-cell recordings of light-induced current (LIC) from isolated Drosophila ommatidia of the trp^{P343} mutant. Left panel: Control responses (middle trace) to a train of orange light pulses (top trace) of constant intensity (⁻log 1=3.0, n=5). The bottom 2 traces show waveforms of the LICs in a faster time scale. Right panel: Paradigm of the left panel was repeated, except that Compound IX (500 μM) was applied as indicated (n=5). B. Averaged peak amplitude of the light responses (such as in (A)) plotted as a function of time (error bars are SEM, n=5). C. The paradigm of (A) was repeated, except that wt flies were used and a larger dark interval was interposed after the second light pulse (n=5). D. Histogram depicting averaged and normalized peak amplitude of light responses after 6 min in the dark following application of Compound IX-containing solutions (Compound IX, right bar) vs. samples lacking Compound IX (control, left; error bars are SEM, n=5).

FIG. 8: Compound IX inhibits TRPM7 channel expressed in HEK cells. A. The paradigm of FIG. 2 was repeated, except that HEK cells expressing TRPM7 were used and voltage ramps from 100 mV to 100 mV within is were applied every 5 s. Compound IX (500 μM) caused a dramatic decrease in the TRPM7 mediated current. Top and bottom panels for A and C are as described for FIG. 3 (n=8). B. Measurements of blocking efficiency of Compound IX as a function of concentration (error bars are SEM, n≧5). C. Control showing no effect of Compound IX when TRPM7 channel was not expressed (n=5). D. Histogram showing maximal blocking effect obtained for the compounds.

FIG. 9: Effect of Compound IX on synaptic vesicle release in CA3-CA1 primary hippocampal cultures. A. Experimental protocol used to determine number of presynaptic vesicles evoked by simple spikes (30 APs@1 Hz) using FM4-64 dye. Compound IX was applied for 10 min before second dye loading. B. Representative fluorescent images following stimulation with 30 APs@1 Hz in mature CA3-CA1 hippocampal neurons expressing TRPM7-GFP fusion protein before (left panel) and 10 min after (right panel) application of 500 μM Compound IX. Fluorescence intensities (arbitrary units) were coded using a pseudocolor transformation shown on the right. C. The same as in (B), but for control neurons lacking TRPM7-GFP. D. Histograms of ΔF of boutons in TRPM7-GFP-expressing neurons before (thin line) and after (thick line) Compound IX application (500 μM) from the experiment shown in (B). ΔF median changed from 73 to 75 arbitrary units (a.u), and the number of FM-detectable boutons decreased from 578 to 318. E. Histograms of ΔF of boutons in control neurons before (thin line) and after (thick line) Compound IX application (500 μM) from the experiment shown in (C). ΔF median changed from 88 to 87 arbitrary units, and FM-detectable boutons changed from 764 to 845. F. Average magnitude of Compound IX effect on presynaptic strength for TRPM7-GFP and WT neurons (error bars are SEM, n=4, P<0.001).

FIG. 10: Compound IX exhibits a significant protective effect from brain damage. A. 10 different tasks were used to evaluate motor ability, balancing and alertness (the NSS score) at different times after trauma (n=20 for each group). Compound IX was injected subcutaneously, while saline injection was used as a control. B. Differences in NSS (dNSS) between control and Compound IX-treated mice.

### DETAILED DESCRIPTION OF THE INVENTION

In one embodiment, the present invention is directed to use of alkyl phenol and monocyclic monoterpene-derived compounds in the treatment of neuronal damage and in inhibiting activity of TRPC and non-thermo-TRPM channels. In another embodiment, the present invention provides use of bicyclic monoterpene-derived compounds in activating TRPC and non-thermo-TRPM channels.

In another embodiment, the present invention provides a composition including one or more compounds of Formula I:

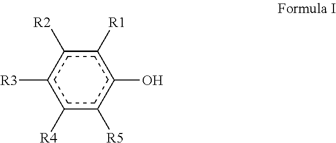

wherein the structure

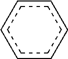

refers to a 6-carbon ring that is either aromatic or has 0, 1, or 2 double bonds;
   for treating or inhibiting neuronal damage,
wherein R¹, R², R³, R⁴, and R⁵ are independently selected from the group consisting of:
   H;
   C₁₋₄ alkyl that is unsubstituted or substituted with 1-4 substituents, wherein the substituents are independently selected from the group consisting of I; Br; Cl; F; nitro; amino; —OH; ═O; C₁-C₃ alkoxy; C₁-C₃ thioalkoxy; C₁-C₃ haloalkyl with 1 to 6 halogen substitutions; C₁-C₃ haloalkoxy with 1 to 6 halogen substitutions; and C₁-C₃ heteroalkyl with 1 to 2 heteroatoms selected from the group consisting of O, N and S;
   C₁₋₄ alkoxy that is unsubstituted or substituted with 1-4 substituents as recited above for C₁₋₄ alkyl;
   CF₃; OH; I; Br; Cl; F; NH₃; and NO₂;
      with the proviso that at least one of R¹, R², R³, R⁴, and R⁵ is C₁₋₄ alkyl that is unsubstituted or substituted as recited above. In another embodiment, a hydrous or anhydrous salt of Formula I is utilized. In another embodiment, the salt is hydrous. In another embodiment, the salt is anhydrous. In another embodiment, a metastable or stable polymorph of Formula I is utilized. In another embodiment, the polymorph is metastable. In another embodiment, the polymorph is stable. Each specific alternative may be considered as a separate embodiment.

In another embodiment, the present invention provides a composition including one or more compounds of Formula II:

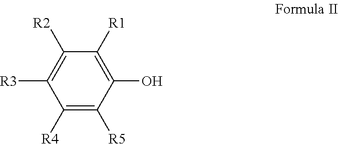

wherein R¹, R², R³, R⁴, and R⁵ are independently selected from the group consisting of:
   H;
   C₁₋₄ alkyl that is unsubstituted or substituted with 1-4 substituents, wherein the substituents are independently selected from the group consisting of I; Br; Cl; F; nitro; amino; —OH; ═O; C₁-C₃ alkoxy; C₁-C₃ thioalkoxy; C₁-C₃ haloalkyl with 1 to 6 halogen substitutions; C₁-C₃ haloalkoxy with 1 to 6 halogen substitutions; and C₁-C₃ heteroalkyl with 1 to 2 heteroatoms selected from the group consisting of O, N and S;
   C₁₋₄ alkoxy that is unsubstituted or substituted with 1-4 substituents as recited above for C₁₋₄ alkyl;
   CF₃; OH; I; Br; Cl; F; NH₃; and NO₂;
      with the proviso that at least one of R¹, R², R³, R⁴, and R⁵ is C₁₋₄ alkyl that is unsubstituted or substituted as recited above. In another embodiment, a hydrous or anhydrous salt of Formula II is utilized. In another embodiment, the salt is hydrous. In another embodiment, the salt is anhydrous. In another embodiment, a metastable or stable polymorph of Formula II is utilized. In another embodiment, the polymorph is metastable. In another embodiment, the polymorph is stable. Each specific alternative may be considered as a separate embodiment.

In another family of embodiments R¹, R², R³, R⁴, and R⁵ are independently selected from the group consisting of H, C₁₋₄ alkyl that is unsubstituted or substituted as recited above, and unsubstituted C₁₋₄ alkoxy.

In certain embodiments, the C₁₋₄ alkyl, if substituted, is substituted with an aldehyde moiety. In other embodiments, R¹, R², R³, R⁴, and R⁵ are independently selected from the group consisting of H, C₁₋₃ alkyl that is unsubstituted or substituted as recited above, and unsubstituted C₁₋₃ alkoxy. In other embodiments, the C₁₋₃ alkyl, if substituted, is substituted with an aldehyde moiety. In still other embodiments, R¹, R², R³, R⁴, and R⁵ are independently selected from the group consisting of H and unsubstituted C₁₋₄ alkyl. In certain other embodiments, R¹, R², R³, R⁴, and R⁵ are independently selected from the group consisting of H and unsubstituted C₁₋₃ alkyl. Each specific alternative may be considered as a separate embodiment.

In another embodiment regarding Formula I, when at least one of R¹, R², R³, R⁴, and R⁵ is substituted or unsubstituted C₁₋₄ alkyl, at least one of the C₁₋₄ alkyl moieties is selected from the group consisting of an isopropenol moiety, an isopropenyl moiety and a tert-Butyl moiety, wherein the branched moiety (i.e. isopropenol, isopropenyl, or tert-Butyl moiety) is unsubstituted or is substituted with 1-4 substituents, wherein the substituents are independently as recited above for C₁₋₄ alkyl.

In other embodiments, at least one of the C₁₋₄ alkyl moieties is selected from the group consisting of an isopropenol moiety and a tert-Butyl moiety. In other embodiments, each branched moiety in the compound is either unsubstituted or is substituted with no more than 3 substituents. In other embodiments, each branched moiety in the compound is either unsubstituted or is substituted with no more than 2 substituents. In other embodiments, each branched moiety in the compound is unsubstituted or is substituted with no more than 1 substituent. In other embodiments, every branched moiety in the compound is unsubstituted. Each possibility may be considered as representing a separate embodiment.

In various embodiments, a branched moiety may assume any position on the cyclic structure relative to the —OH moiety. In some embodiments, the branched moiety is either ortho- or meta- on the cyclic structure relative to the —OH. Each possibility may be considered as representing a separate embodiment.

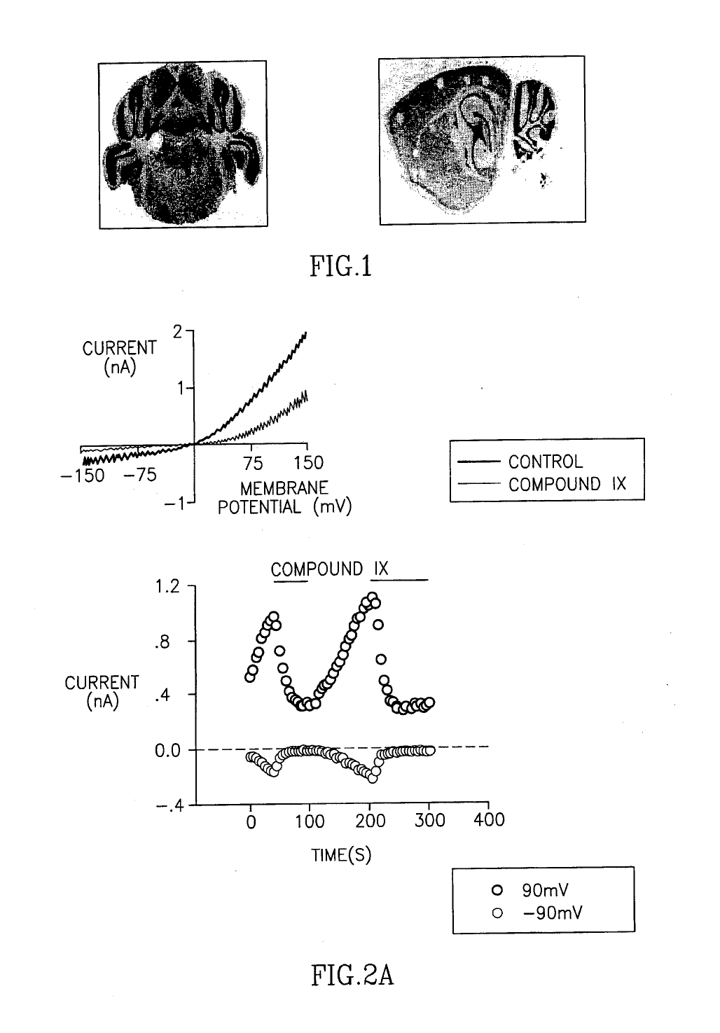

As provided herein, OH-substituted aromatic monocyclic monoterpenoid compounds such as Compound IX, Compound XIII, and Compound XIV inhibit activity of both (a) TRPL and other TRPC-family channels and (b) non-thermo-TRPM channels. Compound IX and Compound XIV contain an unsubstituted branched moiety. Since these channels are highly expressed in mammalian brain (FIG. 1), and they play a key role in neuronal death in response to oxidative stress and excitotoxic signal pathways, agents that inhibit these channels might be expected to prevent death of CNS neurons during neuronal damage, e.g. as mediated by acute brain injury; this was in fact found to be the case. Thus, pungent alkyl phenol compounds such as the monoterpene-derived compounds described herein are considered to be efficacious in treating and inhibiting neuronal damage and acute neurodegeneration. The TRP inhibitors identified herein are also activators of TRPV3 (Vogt-Eisele A K et al, 2007).

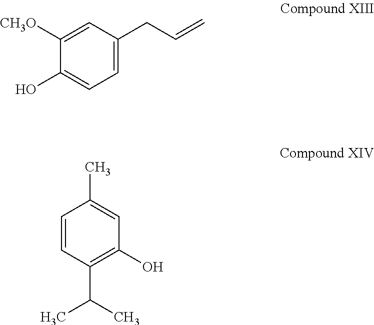

In some embodiments, a ring structure of a compound of the present invention includes, in addition to an above-described branched moiety, a C₁₋₄ alkyl moiety that is unsubstituted or is substituted as recited above for the C₁₋₄ alkyl moiety. In yet other embodiments, a ring structure of a compound of the present invention includes a branched moiety and a C₁₋₃ alkyl moiety that is unsubstituted or is substituted as recited above. In other embodiments, a ring structure of a compound of the present invention includes a branched moiety and a C₁₋₂ alkyl moiety that is unsubstituted or is substituted as recited above. In other embodiments, a ring structure of a compound of the present invention includes a branched moiety and a C₁ alkyl (methyl) moiety that is unsubstituted or is substituted as recited above. In other embodiments, the alkyl moiety is unsubstituted. Each possibility may be considered as representing a separate embodiment.

In specific embodiments, the alkyl moiety may assume various spatial orientations and may be ortho, meta, or para relative to the branched moiety. In particularly preferred embodiments, the alkyl moiety is para relative to the branched moiety. Each specific alternative may be considered as a separate embodiment.

In still other embodiments, the alkyl moiety may be ortho, meta, or para relative to the —OH moiety. In a particularly preferred embodiment, the alkyl moiety is ortho- or meta-relative to the —OH moiety. Each specific alternative may be considered as a separate embodiment.

In a specific embodiment, the compound used in a method of the invention is Compound IX. In another specific embodiment, the compound is Compound XIV. In another specific embodiment, the compound is Compound XIII. Each specific alternative may be considered as a separate embodiment.

In another specific embodiment, the invention includes Formula V (4-Hydroxy-3-methoxybenzaldehyde):

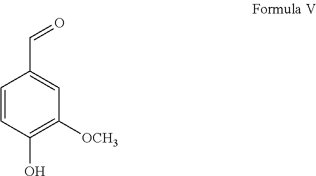

In another embodiment, the present invention includes Formula VI (3-Ethoxy-4-hydroxybenzaldehyde):

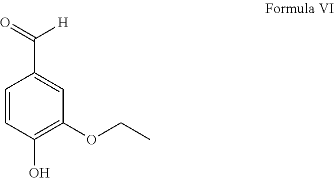

In other embodiments, the present invention provides a composition including a compound of Formula III:

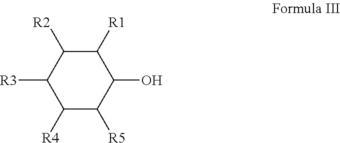

wherein R¹, R², R³, R⁴, and R⁵ are independently selected from the group consisting of:
   H;
   C₁₋₄ alkyl which is unsubstituted or substituted with 1-4 substituents, wherein the substituents are independently selected from the group consisting of I; Br; Cl; F; nitro; amino; —OH; ═O; C₁-C₃ alkoxy; C₁-C₃ thioalkoxy; C₁-C₃ haloalkyl with 1 to 6 halogen substitutions; C₁-C₃ haloalkoxy with 1 to 6 halogen substitutions; and C₁-C₃ heteroalkyl with 1 to 2 heteroatoms selected from the group consisting of O, N and S;
   C₁₋₄ alkoxy that is unsubstituted or substituted with 1-4 substituents as recited above for C₁₋₄ alkyl;
   CF₃; OH; I; Br; Cl; F; NH₃; and NO₂;
with the proviso that at least one of R¹, R², R³, R⁴, and R⁵ is C₁₋₄ alkyl that is unsubstituted or substituted as recited above;
   for treating or inhibiting neuronal damage. In another embodiment, a hydrous or anhydrous salt of Formula III is utilized. In another embodiment, the salt is hydrous. In another embodiment, the salt is anhydrous. In another embodiment, a metastable or stable polymorph of Formula III is utilized. In another embodiment, the polymorph is metastable. In another embodiment, the polymorph is stable. Each specific alternative may be considered as a separate embodiment.

In other embodiments of Formula III, R¹, R², R³, R⁴, and R⁵ are independently selected from the group consisting of H, C₁₋₄ alkyl that is unsubstituted or substituted as recited above, and unsubstituted C₁₋₄ alkoxy. In certain preferred embodiments, the C₁₋₄ alkyl, if substituted, is substituted with an aldehyde moiety. In other embodiments, R¹, R², R³, R⁴, and R⁵ are independently selected from the group consisting of H, C₁₋₃ alkyl that is unsubstituted or substituted as recited above, and unsubstituted C₁₋₃ alkoxy. In certain preferred embodiments, the C₁₋₃ alkyl, if substituted, is substituted with an aldehyde moiety. In certain preferred embodiments, R¹, R², R³, R⁴, and R⁵ are independently selected from the group consisting of H and unsubstituted C₁₋₄ alkyl. In certain preferred embodiments, R¹, R², R³, R⁴, and R⁵ are independently selected from the group consisting of H and unsubstituted C₁₋₃ alkyl. Each specific alternative may be considered as a separate embodiment.

In yet other embodiments of Formula III, when at least one of R¹, R², R³, R⁴, and R⁵ is substituted or unsubstituted C₁₋₄ alkyl, at least one of the C₁₋₄ alkyl moieties is selected from the group consisting of an isopropenol moiety, an isopropenyl moiety and a tert-Butyl moiety, wherein the branched moiety is unsubstituted or is substituted with 1-4 substituents, wherein the substituents are independently as recited above for C₁₋₄ alkyl. In other embodiments, at least one of the C₁₋₄ alkyl moieties is selected from the group consisting of an isopropenol moiety and a tert-Butyl moiety. In other embodiments, each branched moiety in the compound is unsubstituted or is substituted with no more than 3 substituents. In other embodiments, each branched moiety in the compound is unsubstituted or is substituted with no more than 2 substituents. In other embodiments, each branched moiety in the compound is unsubstituted or is substituted with no more than 1 substituent. In other embodiments, each branched moiety in the compound is unsubstituted. Each specific alternative may be considered as a separate embodiment.

In other embodiments, the branched moiety may be in any position on the cyclic structure relative to the —OH moiety. In other embodiments, the branched moiety is either ortho- or meta- on the cyclic structure relative to the —OH. Each specific alternative may be considered as a separate embodiment.

In other embodiments, the compound used in a method of the invention is Compound X. In other embodiments, the compound is Compound XVI. Each specific alternative may be considered as a separate embodiment.

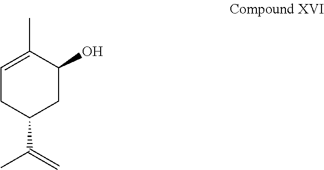

As provided herein, OH-substituted non-aromatic monocyclic monoterpenoid compounds such as Compound X and Compound XVI inhibit activity of TRPC and non-thermo-TRPM channels. Compound X and Compound XVI contain an unsubstituted branched moiety. Thus, pungent compounds such as the non-aromatic monoterpene-derived compounds described herein are useful in treating and inhibiting neuronal damage.

In other embodiments, the present invention provides a composition including one or more compounds of Formula VIII:

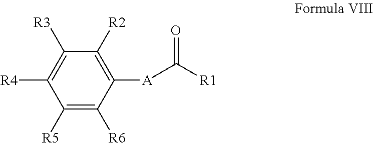

wherein R¹ is selected from the group consisting of H and —OH;
A is a C₁₋₄ alkyl which is either unsubstituted or substituted with 1-4 substituents, wherein the substituents are independently selected from the group consisting of I; Br; Cl; F; nitro; amino; —OH; ═O; C₁-C₃ alkoxy; C₁-C₃ thioalkoxy; C₁-C₃ haloalkyl with 1 to 6 halogen substitutions; C₁-C₃ haloalkoxy with 1 to 6 halogen substitutions; and C₁-C₃ heteroalkyl with 1 to 2 heteroatoms selected from the group consisting of O, N and S; and
R², R³, R⁴, R⁵, and R⁶ are independently selected from the group consisting of:
   H;
   C₁₋₄ alkyl which is unsubstituted or substituted with 1-4 substituents, wherein the substituents are independently selected from the group consisting of I; Br; Cl; F; nitro; amino; —OH; ═O; C₁-C₃ alkyl; C₁-C₃ alkoxy; C₁-C₃ thioalkoxy; C₁-C₃ haloalkyl with 1 to 5 halogen substitutions; C₁-C₃ haloalkoxy with 1 to 5 halogen substitutions; and C₁-C₃ heteroalkyl with 1 to 3 heteroatoms selected from the group consisting of O, N and S;
   C₁₋₄ alkoxy that is unsubstituted or substituted with 1-4 substituents as recited above for C₁₋₄ alkyl;
   CF₃; OH; I; Br; Cl; F; NH₃; and NO₂;
      for treating or inhibiting neuronal damage. In another embodiment, a hydrous or anhydrous salt of Formula VIII is utilized. In another embodiment, the salt is hydrous. In another embodiment, the salt is anhydrous. In another embodiment, a metastable or stable polymorph of Formula VIII is utilized. In another embodiment, the polymorph is metastable. In another embodiment, the polymorph is stable. Each specific alternative may be considered as a separate embodiment.

In another embodiment of Formula VIII, R₂, R₃, R₄, R₅, and R₆ are independently selected from the group consisting of H, unsubstituted or substituted methyl, OH, I, Br, Cl, F, NH₃, and NO₂. In another embodiment, R₂, R₃, R₄, R₅, and R₆ are independently selected from the group consisting of H and unsubstituted methyl. In other embodiments, R₂, R₃, R₄, R₅, and R₆ are all H. Each specific alternative may be considered as a separate embodiment.

In other embodiments of Formula VIII; A is C₁₋₃ alkyl that is unsubstituted or substituted with 1-4 substituents as recited above. In another embodiment, A contains 0-3 substituents. In another embodiment, A contains 0-2 substituents. In other embodiments, A contains 0-1 substituents. In other embodiments, A is an unsubstituted alkyl moiety. In preferred embodiments, A is an unsubstituted C₁₋₃ alkyl moiety. In other preferred embodiments, A is a mono-unsaturated alkyl moiety. In a particularly preferred embodiment, A is ethylene. Each specific alternative may be considered as a separate embodiment.

In another particularly preferred embodiment, the structure formed by AC═OR¹ is Formula VII:

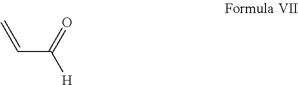

As provided herein, Compound XII and related aromatic compounds described herein inhibit activity of TRPC and non-thermo-TRPM channels. Thus, pungent aromatic compounds containing a mono-unsaturated, aldehyde-substituted alkyl ring substituent are useful in treating and inhibiting neuronal damage.

In other embodiments, the present invention provides a composition including a compound of Formula IV:

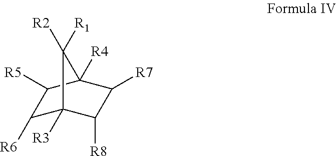

wherein R¹, R², R³, and R⁴ are independently selected from the group consisting of methyl which is unsubstituted or substituted with 1-3 substituents, wherein the substituents are independently selected from the group consisting of I; Br; Cl; F; nitro; amino; —OH; and ═O;
wherein R⁵, R⁶, and R⁷ are independently selected from the group consisting of:
   H;
   Cl_{—}2 alkyl which is unsubstituted or substituted with 1-3 substituents, wherein the substituents are independently selected from the group consisting of I; Br; Cl; F; nitro; amino; —OH; and ═O;
   C₁₋₂ alkoxy that is unsubstituted or substituted with 1-4 substituents as recited above for C₁₋₄ alkyl;
   CF₃; OH; I; Br; Cl; F; NH₃; and NO₂; and
wherein R⁸ is selected from the group consisting of —OH and ═O,
   for treating pruritus secondary to an inflammatory disorder selected from the group consisting of psoriasis, eczema, sunburn, allergic contact dermatitis (ACD), and physical urticaria; treating a disease selected from the group consisting of Darier's and Hailey Hailey's diseases; or reducing the incidence of basal cell carcinoma (BCC). In another embodiment, a hydrous or anhydrous salt of Formula IV is utilized. In another embodiment, the salt is hydrous. In another embodiment, the salt is anhydrous. In another embodiment, a metastable or stable polymorph of Formula IV is utilized. In another embodiment, the polymorph is metastable. In another embodiment, the polymorph is stable. Each specific alternative may be considered as a separate embodiment.

In other embodiments, when at least 1 of R⁵, R⁶, and R⁷ is unsubstituted or substituted alkyl or alkoxy, the alkyl or alkoxy contains 1 carbon atom (i.e. is unsubstituted or substituted methyl or methyoxy.

In yet other embodiments, at least 1 of R₁, R², R³, and R⁴ is unsubstituted methyl. In other embodiments, at least 2 of R¹, R², R³, and R⁴ are unsubstituted methyl. In another, preferred, embodiment, R¹, R², R³, and R⁴ are all unsubstituted methyl. Each specific alternative may be considered as a separate embodiment.

In another embodiment, at least 1 of R⁵, R⁶, and R⁷ is H. In another embodiment, at least 2 of R⁵, R⁶, and R⁷ are H. In another embodiment, R⁵, R⁶, and R⁷ are all H. Each specific alternative may be considered as a separate embodiment.

In some embodiments, R⁸ is —OH. In other embodiments, R⁸ is ═O. Each specific alternative may be considered as a separate embodiment.

In other embodiments, the compound used in a method of the invention is Compound XVII. In another embodiment, the compound is Compound XI. Each specific alternative may be considered as a separate embodiment.

As provided herein, —OH— or ═O-substituted bicyclic monoterpenoid compounds such as Compound XVII and Compound XI activate TRPC and non-thermo-TRPM channels. Activation of these channels is useful in treating pathogenic pruritus secondary to inflammatory disorders including inter alia psoriasis, eczema, sunburn, allergic contact dermatitis (ACD), and physical urticarias; treating Darier's and Hailey Hailey's diseases; and reducing the incidence of basal cell carcinoma (BCC) (Stokes et al; Beck et al; Nilius et al).

Thus, bicyclic monoterpene-derived compounds such as those described herein are efficacious in activating TRPC and non-thermo-TRPM channels.

In other embodiments, any of the R moieties described above is alkyl that is unsubstituted or substituted with 1-4 substituents, the substituents include alkoxy, thioalkoxy, haloalkyl, haloalkoxy, or heteroalkyl, and the size of each substituent is limited to 2 carbon atoms. In other embodiments, the size of each substituent is limited to 1 carbon atom. Those of skill in the art will understand that in each case, the number of halogen substitutions will be limited to 3 times the number of carbon atoms. Each specific alternative may be considered as a separate embodiment.

In other embodiments, when any of the R moieties described above is C₁₋₄ alkyl that is unsubstituted or substituted with 1-4 substituents, the substituents are independently selected from the group consisting of I; Br; Cl; F; nitro; amino; —OH; and ═O.

In other embodiments, when any of the R moieties described above is alkyl that is unsubstituted or substituted, the number of substituents is limited to 3 per alkyl moiety. In other embodiments, the number of substituents is limited to 2 per alkyl moiety. In certain preferred embodiments, the number of substituents is limited to 1 per alkyl moiety. In certain preferred embodiments, all alkyl moieties in the compound are unsubstituted or substituted with an aldehyde moiety. In certain preferred embodiments, all alkyl moieties in the compound are unsubstituted. Each specific alternative may be considered as a separate embodiment.

In another embodiment, when any of the R moieties described above is alkyl that is unsubstituted or substituted, the number of carbon atoms is limited to 3 per alkyl moiety. In another embodiment, the number of carbon atoms is limited to 2 per alkyl moiety. In certain embodiments, the alkyl moiety is unsubstituted or substituted methyl. Each specific alternative may be considered as a separate embodiment.

As provided herein, methods of the present invention are useful in treating brain damage, e.g. that caused by neuronal damage. “Neuronal damage” as used herein refers to neuronal damage that may occur for example as a result of acute brain injury. In one embodiment, the neuronal damage of a subject treated by a method of the present invention is mediated by hypoxia. In another embodiment, the neuronal damage is mediated by oxygen free radicals downstream of an excitotoxic signal pathway. In a preferred embodiment, the neuronal damage results from acute traumatic brain injury (TBI). It will be appreciated that the neuronal damage may be secondary to any other cause known in the art. Each specific alternative may be considered as a separate embodiment.

### Pharmaceutical Compositions and Methods of the Present Invention

In other embodiments, the present invention provides a pharmaceutical composition for treating or inhibiting neuronal damage, including a pharmaceutically acceptable carrier and as an active ingredient the compound of Formula II.

In yet other embodiments, the present invention provides a pharmaceutical composition for treating or inhibiting neuronal damage, including a pharmaceutically acceptable carrier and as an active ingredient the compound of Formula III.

In other embodiments, the present invention provides a pharmaceutical composition for treating or inhibiting neuronal damage, including a pharmaceutically acceptable carrier and as an active ingredient the compound of Formula VIII.

In still other embodiments, the present invention provides a pharmaceutical composition for inhibiting activity of a TRPC channel, including a pharmaceutically acceptable carrier and as an active ingredient the compound of Formula II.

In still other embodiments, the present invention provides a pharmaceutical composition for inhibiting activity of a TRPC channel, including a pharmaceutically acceptable carrier and as an active ingredient the compound of Formula III.

In yet other embodiments, the present invention provides a pharmaceutical composition for inhibiting activity of a TRPC channel, including a pharmaceutically acceptable carrier and as an active ingredient the compound of Formula VIII.

In yet other embodiments, the present invention provides a pharmaceutical composition for inhibiting activity of a non-thermo-TRPM channel, including a pharmaceutically acceptable carrier and as an active ingredient the compound of Formula II.

In other embodiments, there is provided a pharmaceutical composition for inhibiting activity of a non-thermo-TRPM channel, including a pharmaceutically acceptable carrier and as an active ingredient the compound of Formula III.

In other embodiments, the present invention provides a pharmaceutical composition for inhibiting activity of a non-thermo-TRPM channel, including a pharmaceutically acceptable carrier and as an active ingredient the compound of Formula VIII.

In other embodiments, there is provided a pharmaceutical composition for activating a TRPC channel, including a pharmaceutically acceptable carrier and as an active ingredient the compound of Formula IV.

In other embodiments, there is provided a pharmaceutical composition for treating pruritus secondary to an inflammatory disorder selected from the group consisting of psoriasis, eczema, sunburn, allergic contact dermatitis, and physical urticaria, including a pharmaceutically acceptable carrier and as an active ingredient the compound of Formula IV.

In other embodiments, there is provided a pharmaceutical composition for treating a disease selected from the group consisting of Darier's and Hailey Hailey's diseases, including a pharmaceutically acceptable carrier and as an active ingredient the compound of Formula IV.

In other embodiments, there is provided a pharmaceutical composition for reducing the incidence of basal cell carcinoma (BCC), including a pharmaceutically acceptable carrier and as an active ingredient the compound of Formula IV.

In another embodiment, the present invention provides use of the compound of Formula II in the preparation of a medicament for treating or inhibiting neuronal damage or inhibiting activity of a TRPC or non-thermo-TRPM channel.

In another embodiment, the present invention provides use of the compound of Formula III in the preparation of a medicament for the treating or inhibiting neuronal damage or inhibiting activity of a TRPC or non-thermo-TRPM channel.

In another embodiment, the present invention provides use of the compound of Formula VIII in the preparation of a medicament for the treating or inhibiting neuronal damage or inhibiting activity of a TRPC or non-thermo-TRPM channel.

In another embodiment, the present invention provides use of the compound of Formula IV in the preparation of a medicament for activating a TRPC channel or non-thermo-TRPM channel.

In another embodiment, the present invention provides use of the compound of Formula IV in the preparation of a medicament for treating pruritus secondary to an inflammatory disorder selected from the group consisting of psoriasis, eczema, sunburn, allergic contact dermatitis, and physical urticaria.

In another embodiment, the present invention provides use of the compound of Formula IV in the preparation of a medicament for treating a disease selected from the group consisting of Darier's and Hailey Hailey's diseases.

In another embodiment, the present invention provides use of the compound of Formula IV in the preparation of a medicament for reducing the incidence of basal cell carcinoma (BCC).

In another embodiment, the present invention provides a method for treating or inhibiting neuronal damage, the method including the step of administering to a subject in need of such treatment a therapeutically effective amount of a compound of Formula II, thereby treating or inhibiting neuronal damage.

In another embodiment, the present invention provides a method for treating or inhibiting neuronal damage, the method including the step of administering to a subject in need of such treatment a therapeutically effective amount of a compound of Formula III, thereby treating or inhibiting neuronal damage.

In another embodiment, the present invention provides a method for treating or inhibiting neuronal damage, the method including the step of administering to a subject in need of such treatment a therapeutically effective amount of a compound of Formula VIII, thereby treating or inhibiting neuronal damage.

In another embodiment, the present invention provides a method for inhibiting signaling by a TRPC channel, the method including the step of administering to a subject in need of such treatment a therapeutically effective amount of a compound of Formula II, thereby inhibiting signaling by a TRPC channel.

In another embodiment, the present invention provides a method for inhibiting signaling by a TRPC channel, the method including the step of administering to a subject in need of such treatment a therapeutically effective amount of a compound of Formula III, thereby inhibiting signaling by a TRPC channel.

In another embodiment, the present invention provides a method for inhibiting signaling by a TRPC channel, the method including the step of administering to a subject in need of such treatment a therapeutically effective amount of a compound of Formula VIII, thereby inhibiting signaling by a TRPC channel.

In another embodiment, the present invention provides a method for inhibiting signaling by a non-thermo-TRPM channel, the method including the step of administering to a subject in need of such treatment a therapeutically effective amount of a compound of Formula II, thereby inhibiting signaling by a non-thermo-TRPM channel.

In another embodiment, the present invention provides a method for inhibiting signaling by a non-thermo-TRPM channel, the method including the step of administering to a subject in need of such treatment a therapeutically effective amount of a compound of Formula III, thereby inhibiting signaling by a non-thermo-TRPM channel.

In another embodiment, the present invention provides a method for inhibiting signaling by a non-thermo-TRPM channel, the method including the step of administering to a subject in need of such treatment a therapeutically effective amount of a compound of Formula VIII, thereby inhibiting signaling by a non-thermo-TRPM channel.

In another embodiment, the present invention provides a method for activating signaling by a TRPC channel, the method including the step of administering to a subject in need of such treatment a therapeutically effective amount of a compound of Formula IV, thereby activating signaling by a TRPC channel.

In another embodiment, the present invention provides a method for activating signaling by a non-thermo-TRPM channel, the method including the step of administering to a subject in need of such treatment a therapeutically effective amount of a compound of Formula IV, thereby inhibiting signaling by a non-thermo-TRPM channel.

In another embodiment, the present invention provides a method for treating pruritus secondary to an inflammatory disorder selected from the group consisting of psoriasis, eczema, sunburn, allergic contact dermatitis, and physical urticaria, the method including the step of administering to a subject in need of such treatment a therapeutically effective amount of a compound of Formula IV, thereby treating pruritus secondary to psoriasis, eczema, sunburn, allergic contact dermatitis, or physical urticaria.

In another embodiment, the present invention provides a method for treating a disease selected from the group consisting of Darier's and Hailey Hailey's diseases, the method including the step of administering to a subject in need of such treatment a therapeutically effective amount of a compound of Formula IV, thereby treating Darier's or Hailey Hailey's disease.

In another embodiment, the present invention provides a method for reducing the incidence of basal cell carcinoma (BCC), the method including the step of administering to a subject in need of such treatment a therapeutically effective amount of a compound of Formula IV, thereby reducing the incidence of BCC.

The pharmaceutical compositions of the present invention include a compound of this invention as an active ingredient and a pharmaceutically acceptable carrier and optionally other therapeutic ingredients. Any suitable route of administration may be employed for providing a mammal, especially a human with an effective dosage of a compound of the present invention. For example, oral, rectal, topical, parenteral, ocular, pulmonary, nasal, and the like may be employed. Dosage forms include tablets, troches, dispersions, suspensions, solutions, capsules, creams, ointments, aerosols, and the like.

The compositions include compositions suitable for oral, rectal, topical, parenteral (including subcutaneous, intramuscular, and intravenous), ocular (ophthalmic), pulmonary (nasal or buccal inhalation), or nasal administration, although the most suitable route in any given case will depend on the nature and severity of the conditions being treated and on the nature of the active ingredient. In certain preferred embodiments, compounds of the present invention are administered by direct injection to the brain. In certain other preferred embodiments, the compounds are administered by a route selected from the group consisting of oral administration and topical administration to the cranium. They may be conveniently presented in unit dosage form and prepared by any of the methods well-known in the art of pharmacy.

For administration by inhalation, the compounds of the present invention may be conveniently delivered in the form of an aerosol spray presentation from pressurized packs or nebulisers. The compounds may also be delivered as powders which may be formulated and the powder composition may be inhaled with the aid of an insufflation powder inhaler device. The preferred delivery system for inhalation is a metered dose inhalation (MDI) aerosol, which may be formulated as a suspension or solution of a compound of this invention in suitable propellants, such as fluorocarbons or hydrocarbons.

Suitable topical formulations of a compound of this invention include transdermal devices, aerosols, creams, ointments, lotions, dusting powders, and the like.

### TRPC Channels

TRPC channels are well known in the art, and are described, inter alia, in Nilius B et al (ibid). TRPC channel family members are set forth in below in Table 2 from Nilius B et al, ibid.

In other embodiments, the TRPC channel of the present invention is selected from the group consisting of TRPC1, TRPC2, TRPC3, TRPC4, TRPC5, TRPC6, and TRPC7. In another embodiment, in the case of humans, the TRPC channel is selected from the group consisting of TRPC1, TRPC3, TRPC4, TRPC5, TRPC6, and TRPC7In another embodiment, the TRPC channel is selected from the group that is a subset of these 7 receptors. Each possibility represents a separate embodiment.

TABLE 2
TRPC channel family members.

| | Ensembl Gene Identification |
|---|---|
| Channel/SEQ ID No. | No/NCBI Accession No. |
| TRPC1/SEQ ID No: 1 | ENSG00000144935/NP_003295. |
| TRPC2/SEQ ID No: 2 | ENSMUSG00000070425/NP_035774 |
| TRPC3/SEQ ID No: 3-4 | ENSG00000138741/NP_001124170 |
| | (isoform a), NP_003296 (isoform b) |
| TRPC4/SEQ ID No: 5-10 | ENSG00000100991 |
| | isoform alpha-NP_057263 |
| | isoform beta-NP_001129427 |
| | isoform gamma-NP_001129428 |
| | isoform delta-NP_001129429 |
| | isoform epsilon-NP_003297 |
| | isoform zeta-NP_001129430 |
| TRPC5/SEQ ID No: 11 | ENSG00000072315/NP_036603 |
| TRPC6/SEQ ID No: 12 | ENSG00000137672/NP_004612 |
| TRPC7/SEQ ID No: 13 | ENSG00000069018/NP_065122 |

### Thermo- and Non-Thermo-TRPM Channels

TRPM channels are well known in the art, and are described, inter alia, in Nilius B et al (ibid). Some characteristics of TRPM channel family members are set forth in below in Table 3 from Nilius B et al, ibid. “Non-thermo-TRPM channel” as used herein refers to a TRPM channel not activated by heat or cold, and includes TRPM1, TRPM3, TRPM6, and TRPM7. Thus, in another embodiment, the TRPM channel of the present invention is selected from the group consisting of TRPM1 (SEQ ID No: 14), TRPM3 (SEQ ID No: 15), TRPM6 (SEQ ID No: 16), and TRPM7 (SEQ ID No: 17). In another embodiment, the non-thermo-TRPC channel is selected from the group that is a subset of these 4 receptors. Each specific alternative may be considered as a separate embodiment.

TABLE 3
TRPM channel family members and the Accession Numbers of their
corresponding mRNA.

| Channel/Ensembl and | | Conduct- | Proposed |
|---|---|---|---|
| NCBI Gene ID No./ | Selectivity | ance, | Activation |
| SEQ ID No (if any) | P_{Ca}/P_{Na} | pS | Mechanisms |
| TRPM1 | ND | ND | Translocation, MITF- |
| ENSG00000134160 | | | induced expression |
| NM_002420/14 | | | |
| TRPM2 | 0.5-1.6 | 52-80 | ADP-ribose, cADPR, |
| ENSG00000142185 | | | NAD, heat, H₂O₂ |
| NM_003307 | | | and other |
| | | | ROS, inhibition of |
| | | | PARP-1 |
| TRPM3 | 1.6-2.0 | 65(Ca²⁺)- | Cell swelling, store |
| ENSG00000083067 | | 130 | depletion, D- |
| NM_206945/15 | | | erythrosphingosine |
| TRPM4 | Selective | 25 | Elevated [Ca²⁺]ᵢ, ATP, |
| ENSG00000130529 | for | | PKC, decavanadate, |
| NM_017636 | monovalent | | voltage dependent; |
| | cations | | heat, PI(4,5)P₂, BTP2 |
| TRPM5 | Selective | 16-25 | Elevated [Ca²⁺]ᵢ, |
| ENSG00000070985 | for | | PI(4,5)P₂, |
| NM_014555 | monovalent | | voltage dependent, |
| | cations | | heat |
| TRPM6 | P_{Mg}/P_{Na} ~6 | ND | Decreased [Mg²⁺]ᵢ, |
| ENSG00000119121 | | | 2-APB |
| NM_017662/16 | | | |
| TRPM7 | 3 | 40-105 | Decreased [Mg²⁺]ᵢ, Mg- |
| ENSG000000 | | | ATP, PI(4,5)P₂, cAMP, |
| NM_017672/17 | | | G protiens, shear stress, |
| | | | memebrane |
| | | | translocation |
| TRPM8 | 1-3 | 83 | Depolarization, cold (8- |
| ENSG000000144481 | | | 28° C.), menthol, |
| NM_024080 | | | icilin, Ca²⁺, increased |
| | | | intracellular pH, |
| | | | PI(4,5)P₂ |

### Experimental Details

### Materials and Experimental Methods

### Tissue Homogenation and In Situ Hybridization

Tissue homogenation was performed as described in the document Allen Brain Atlas ABA Data Production Processes, © 2004-2006 Allen Institute for Brain Science. In situ hybridization was carried out using Ambion's Mega Shortscript Kit (Cat # AM1354) according to standard protocol.

### Description of Model System

The Drosophila photoreceptor cells constitute a native tissue in which two members of the TRPC subfamily, TRP and TRP-like (TRPL) are accessible to whole cell recordings and their physiological functions as the light-activated channels is well defined in vivo (Minke et al, 2002). Studying TRPC channel activation and inhibition by pharmacological agents in the Drosophila eye offers several advantages because of the power of Drosophila molecular genetics. The existence of null mutants in which either the TRP (trp^{P343}) or TRPL (trpl³⁰²) are missing allows studying each of the 2 light-activated channels in isolation. Unlike mammalian TRPC channels heterologously expressed in tissue culture cells, TRPL channels expressed in Drosophila Schneider 2 (S2) cells exhibit similar biophysical properties to the native channel, except that the expressed channels are constitutively active, unlike the native channels that are closed in the dark (Parnas et al, 2007). Therefore, the S2 expression system enables studying the TRPL channel in isolation from the complex phototransduction cascade of Drosophila. In addition, the results from the expressed TRPL channels can be verified in the native system during light exposure and provide physiological insight into the native system.

### Fly Stocks:

Drosophila white-eyed flies of the following strains were used: wt and trp^{P343}. Flies were raised at 24° C. in a 12 h light/dark cycle.

### Light Stimulation:

A tungsten trans-illumination light (12 V, 100 W halogen lamp, in conjunction with a Schott OG 590 orange edge filter and a KG3 heat filter) provided illumination for stimulation of the photoreceptors in all experiments. Stimulating light was applied via a condenser lens (Carl Zeiss Microlmaging, Inc.) and was attenuated by neutral density filters. The maximal luminous intensity of the unattenuated orange light at the level of the ommatidia was 3.2 mW/cm².

### Cell Culture:

Schneider 2 cells were grown in 25 cm² flasks at 25° C. in Schneider medium (Beit HaEmek biological industries, Israel) supplemented with 10% fetal bovine serum (FBS) and 1% pen-strep reagent. TRPL-eGFP (GenBank accession number NM_{—}165694; SEQ ID No: 18) channels were stably expressed. Human embryonic kidney (HEK) cells were grown in 25 cm² flasks at 37° C. in Dulbecco's Modified Eagle's Medium (DMEM) (Beit Haemek biological industries, Israel) supplemented with 10% FBS, 1% pen-strep and 1% L-Glutamine. TRPM7 (GenBank accession number NM_{—}021450; SEQ ID No: 19; provided by David Clapham) and pIRES-eGFP were transiently expressed using TransIT™ reagent (Mirus).

### Hippocampal Cell Culture:

Primary cultures of hippocampal neurons were prepared as described in Slutsky et al, 2004. The experiments were performed in mature (15-28 days in vitro (DIV)) high density cultures (synaptic density>1.5 synapses/μm² of dendritic surface area). TRPM7-GFP was provided by David Clapham and was transiently expressed using Lipofectamin-2000 reagent (Invitrogen).

### Electrophysiology

### S2 and HEK Cells

Cells were seeded on polylysine-coated plates at a confluence of 25%, 24-72 hours prior to the experiment. 24 hours prior to the experiment, 500 μM CuSO₄ was added to the medium of the S2 cells to induce expression of the channels. Whole-cell, patch-clamp recordings were performed as described (Parnas et al, 2007). Currents were recorded at room temperature using borosilicate patch pipettes of 5-8 MΩ and an Axopatch 1D (Molecular Devices, Inc.) voltage-clamp amplifier. Voltage-clamp pulses were generated and data captured using a Digidata 1322A interfaced to a computer running the pClamp 9.2 software (Axon Instruments, Inc.). Currents were filtered using the 8-pole low pass Bessel filter of the patch-clamp amplifier at 5 kHz and sampled at 20 kHz. To measure I-V curves with minimal distortions, only cells with low (<10 MΩ) series resistance were used, and series resistance was compensated to ˜80%.

Drosophila ommatidia

Dissociated Drosophila ommatidia were prepared from newly emerged flies (<1 h after eclosion) that were kept in the dark 12-18 h before the experiment. Whole-cell, patch-clamp recordings were performed as described in Peretz et al, 1994. Currents were recorded at 21° C. using borosilicate patch pipettes of 8-10 MO and an Axopatch 200B™ (Molecular Devices, Inc.) voltage-clamp amplifier. Voltage-clamp pulses were generated and data captured using a Digidata 1200™ interfaced to a computer running pClamp 8.0 software (Axon Instruments, Inc.). Currents were filtered using the 8-pole low pass Bessel filter of the patch-clamp amplifier at 5 kHz and sampled at 10 kHz. Series resistance was compensated to ˜80%.

### Solutions:

For S2 cells, the extracellular solution contained in mM: 150 NaCl, 5 KCl, 4 MgCl₂, 10 TES, 25 proline, 5 alanine and 0.5 EGTA. The intracellular solution contained in mM: 130 K-gluconate, 10 TES, 2 MgCl₂, 4 Mg-ATP and 0.4 Na-GTP. For Drosophila ommatidia, the extracellular solution contained in mM: 120 NaCl, 5 KCl, 4 MgSO₄, 1.5 CaCl₂, 10 TES, 25 proline and 5 alanine. The intracellular solution contained in mM: 140 K-gluconate, 10 TES, 2 MgSO₄, 4 Mg-ATP, 0.4 Na-GTP and 1β-NAD. All solutions were titrated to pH 7.15. For HEK cells, the extracellular solution contained in mM: 140 NaCl, 2.8 KCl, 2 CaCl₂, 1 MgCl₂, HEPES and 10 glucose. The intracellular solution contained in mM: 140 Cs-Aspartate, 8 NaCl, 10 EGTA and 10 HEPES. All solutions were titrated to pH 7.2. Cells were perfused via a BPS-8 valve control system (Scientific Instruments) at a rate of ˜30 chamber volumes per minute. Chemicals were applied via the perfusion system.

### Estimation of Synaptic Vesicle Release Based on FM Dye Staining:

Synaptic vesicle release at single synapses was determined by counting the number of presynaptic vesicles turned over by a fixed number of action potentials using activity-dependent FM dye uptake as a marker (Slutsky et al, 2004). During dye loading, action potentials in neurons were initiated by field stimulation and terminals, undergone vesicle exocytosis, were labelled by FM 4-64 (15 μM) presented in the extracellular solution during and 30 sec after electrical stimulation. Extracellular solution during FM loading and unloading procedures contained (in mM): NaCl, 145; KCl, 3; glucose, 15; HEPES, 10; MgCl₂, 1.2; CaCl₂, 1.2; kynurenic acid 0.5 (Sigma); pH adjusted to 7.4 with NaOH. Kynurenic acid was added in the imaging experiments described herein to prevent recurrent activity through block of excitatory postsynaptic responses during loading and unloading. Following the loading protocol (30 AP@1 Hz), external dye was washed away in Ca²⁺-free solution. To confirm that the fluorescent spots corresponded to release sites, action potentials were evoked at frequency of 2 Hz for 4 min during unloading step in order to cause release of dye-filled vesicles. ΔF, the total amount of releasable fluorescence at each bouton (ΔF=F_{loading}−F_{unloading}), is proportional to release probability. Total presynaptic strength (S) has been estimated by the product of these 2 parameters (S=ΔF×N), where N is the number of active terminals (defined as terminals that release at least 1 vesicle during loading protocol) per FM image.

### Functional Imaging and Analysis:

Images were taken using an Olympus (FV300) confocal laser inverted microscope. The 488 nm line of the argon laser was used for excitation, and the emitted light was filtered through a 510-530 nm band pass filter for GFP and 660 nm long pass filter for FM 4-64 and detected by a photomultiplier. A 60×1.2 NA water-immersion objective was used for imaging. A confocal aperture was partially open and image resolution was 92 inn/pixel. The photomultiplier gain was adjusted to maximize the signal/noise ratio without causing saturation by the strongest signals. The image after FM dye unloading was subtracted from the initial image; thus, only terminals containing activity-dependent releasable FM dye (˜90% of total staining) were analyzed. FM positive puncta were selected for further analysis using custom scripts written in ImagePro Plus™ (Media Cybernetics, Carlsbad, Calif.) and MATLAB™ (Mathworks, Natick, Mass.).

### Mouse Model of Brain Injury

Brain injury is known to disrupt the blood-brain barrier and to induce edema and release auto-destructive factors that produce delayed neuronal damage and neuronal cell death. An experimental model of Closed Brain Injury (CHI) in the rat and mouse has been developed and studied mechanistically (Beni-Adani et al 2001; Chen et al 1996). In the present study, CHI was induced under ether anesthesia by allowing a metal rod (333 g) to fall over the exposed skull covering the left hemisphere in the midcornal plane of a 35-45 g Mouse. 15 min later, mice were injected subcutaneously with Compound IX at a dose of 0.751 μg/gr body weight or saline (negative control). Neurobehavioral evaluations were performed over the next 27 d.

### Neurological Severity Scoring System for Head-Injured Mice.

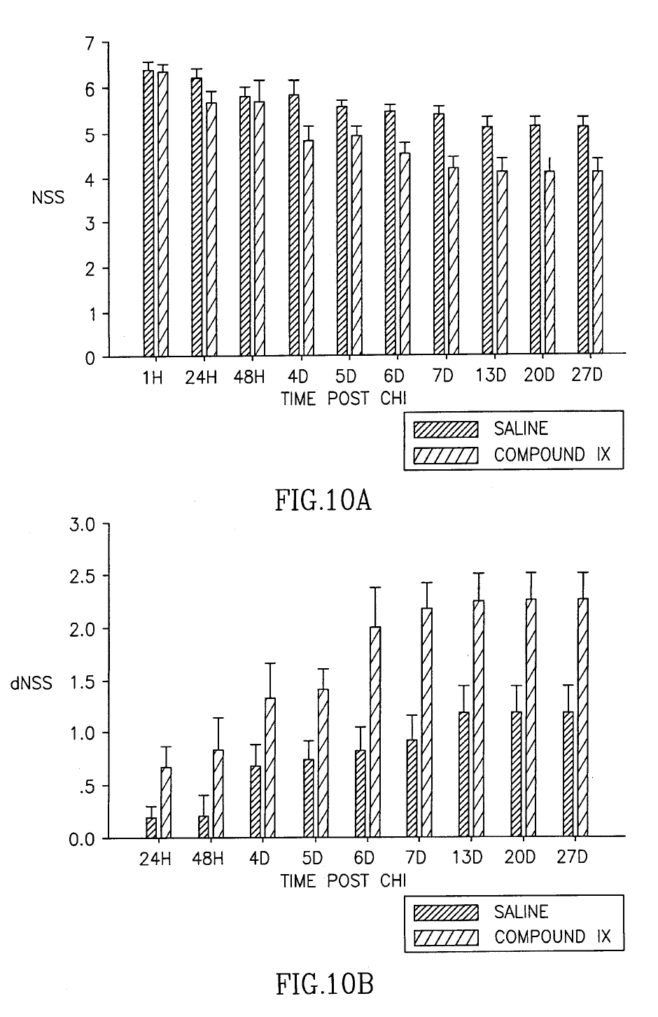

A Neurological Scoring System (NSS) was used to determine ability to perform 10 different behavioral tasks, involving motor ability, balance, and alertness of the mice (Table 4). One point is assigned for failing to perform each task; thus, a normal uninjured mouse scores 0. Injury severity is defined by initial NSS, which is evaluated 1 h post-CHI and referred to as “NSS1.” This value is also a reliable predictor of clinical outcome, with fatal or near-fatal injury corresponding to NSS1 of 7-8; moderate injury to NSS1 of 5-6, and mild injury to NSS1 of <4. Control mice and Compound IX-injected mice did not differ in severity of trauma, as evidenced by their identical initial NSS1 scores (FIG. 10).

TABLE 4
Neurological severity score (NSS) for head-injured mice.

| Task | NSS |
|---|---|
| Presence of mono-or hemiparesis | 1 |
| Inability to walk on a 3-cm-wide beam | 1 |
| Inability to walk on a 2-cm-wide beam | 1 |
| Inability to walk on a 1-cm-wide beam | 1 |
| Inability to balance on a 1-cm-wide beam | 1 |
| Inability to balance on a round stick | 1 |
| Failure to exit a 30-cm diameter circle (for 2 min) | 1 |
| Inability to walk straight | 1 |
| Loss of startle behavior | 1 |
| Loss of seeking behavior | 1 |
| Maximum total | 10 |

### Results

### Example 1

### TRPM7 and TRPC5 are Highly Expressed in Mammalian Brain

Expression of non-thermo-TRPs TRPM7 and TRPC5 was tested in mammalian brain using in situ hybridization. Both of these channels were found to be highly expressed (FIG. 1).

### Example 2

### Compound IX is an Inhibitor of TRPL Channels Expressed in Tissue Culture Cells

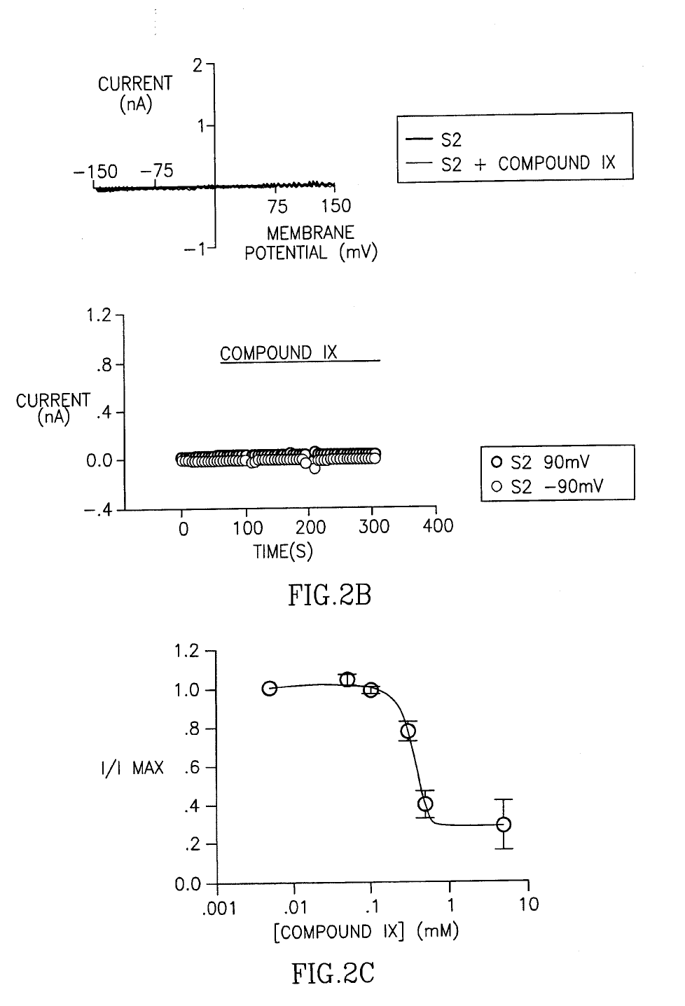

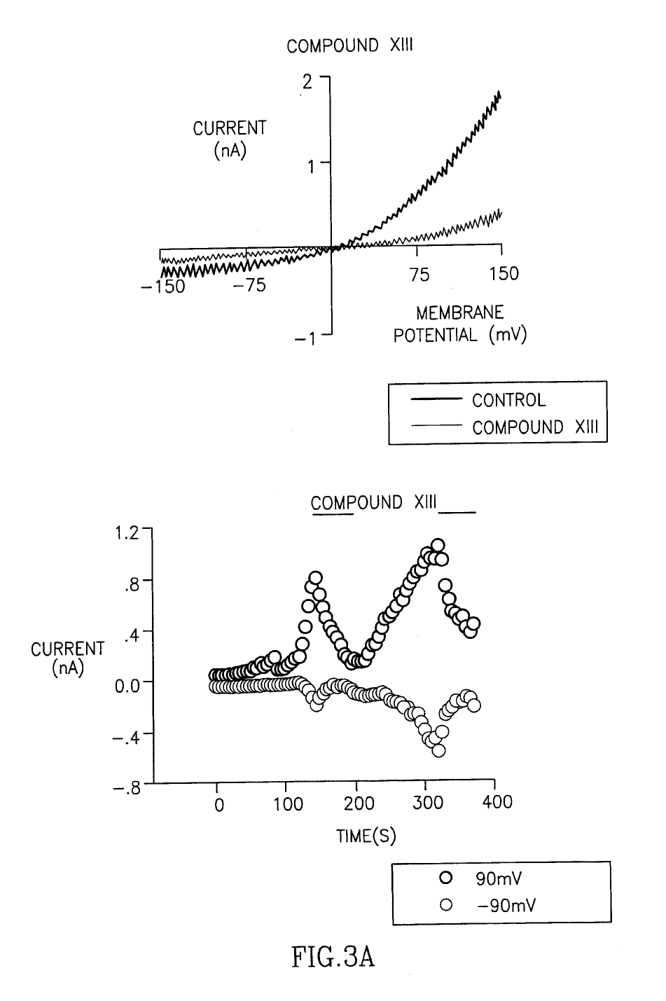

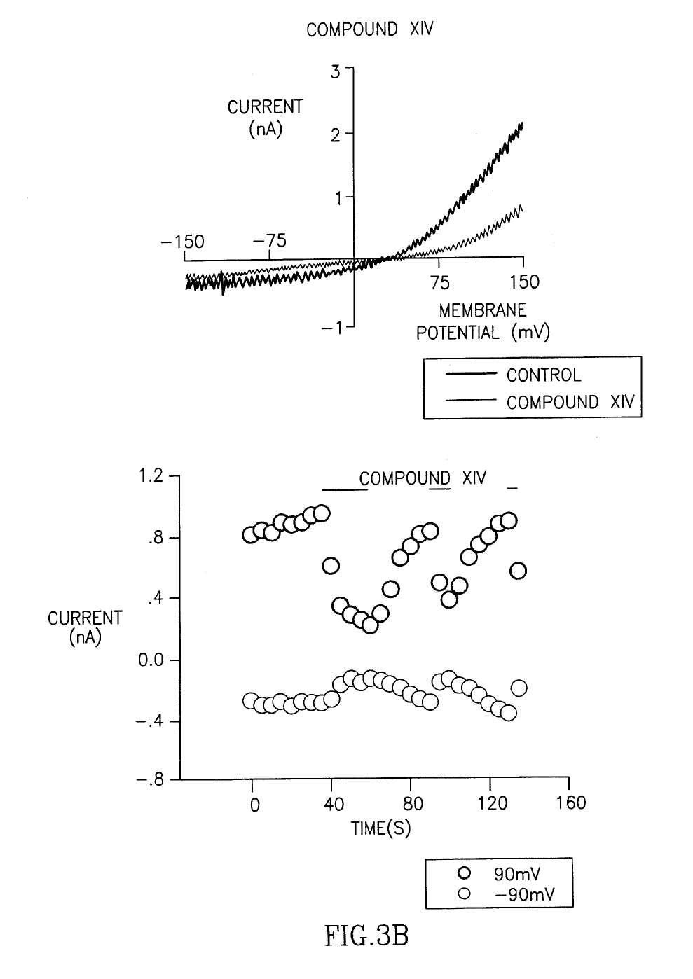

To test whether Compound IX inhibits TRP-like (TRPL) channels, TRPL channels were expressed in S2 cells, and the effect of Compound IX on TRPL-mediated currents was examined. TRPL channels expressed in S2 cells are constitutively active. The level of this constitutive activity varies in individual cells and may show a slow increase of current with time (FIGS. 2-3). The time course of this phenomenon was slow relative to the effect of the tested compounds, and it occurred in the opposite direction of the effect of the inhibitors; thus it did not interfere with measurements of inhibitor activity. To confirm that basal channel activity did not significantly affect experimental results, cells that revealed variable constitutive activity were tested. FIG. 2A (top) shows the typical current-voltage relationships (I-V curves) of active TRPL channels before and after application of Compound IX (Table 1). Large suppression of the current was observed after application of Compound IX (500 μM). Current values at −90 mV and 90 mV obtained from a time series of I-V curves are plotted in FIG. 2A (bottom) as a function of time. Compound IX reversibly blocked the channels, as shown by suppression of both inward and outward current. When TRPL was not expressed, Compound IX had no effect (FIG. 2B). FIG. 2C shows that the IC₅₀ of Compound IX inhibition is 357±14 μM, similar to the potency of Compound IX as an activator of TRPV3. Thus, Compound IX is an inhibitor of the TRPL channel.

TABLE 1
Structures of compounds used herein.

| | | TRPL |
|---|---|---|
| | | Activation/ |
| Name | Formula | Inhibition |
| Compound IX | 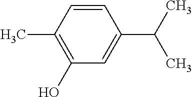 5-Isopropyl-2-methylphenol | Inhibition |
| Compound XIV | 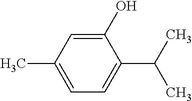 2-Isopropyl-5-methylphenol | Inhibition |
| Compound XIII | 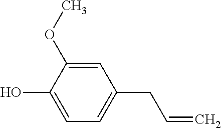 2-Methoxy-4-(2-propenyl)phenol | Inhibition |
| 2-APB | 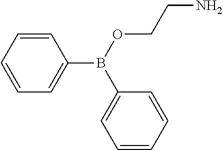 2-Aminoethyl diphenyl borate | Inhibition |
| Compound XII | 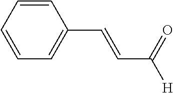 trans-3-Phenyl-2-propenal | Inhibition |
| Compound XV | 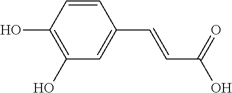 3,4-Dihydroxycinnamic acid | No effect |
| Compound X | 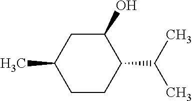 2-Isopropyl-5-methylcyclohexanol | Inhibition |
| Compound XVI | 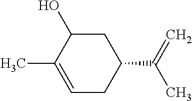 (5R)-5-Isopropenyl-2-methyl-2- cyclohexenol | Inhibition |
| Compound XVII | 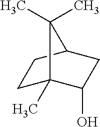 endo-(1R)-1,7,7- Trimethylbicyclo[2.2.1]heptan-2-ol | Activation |
| Compound XI | 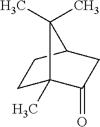 1,7,7-Trimethylbicyclo[2.2.1]heptan-2-one | Activation |

Since Compound IX inhibits TRPL but activates TRPV3, other activators of TRPV3 were also tested for ability to inhibit the TRPL channel. Since all TRPV3 activators require relatively high (<10 mM) concentrations, the same concentrations were used to test their inhibitory effect. Compound XIV and Compound XIII are alkyl phenols with closely related structure to Compound IX (see Table 1) that activate TRPV3 (Xu et al, 2006); their activity against TRPL was tested as described for FIG. 1, except with higher concentrations, namely 3 mM and 1 mM for Compound XIII and Compound XIV, respectively. Both compounds inhibited TRPL, although higher concentrations than Compound IX were required (FIG. 3A-B, respectively).

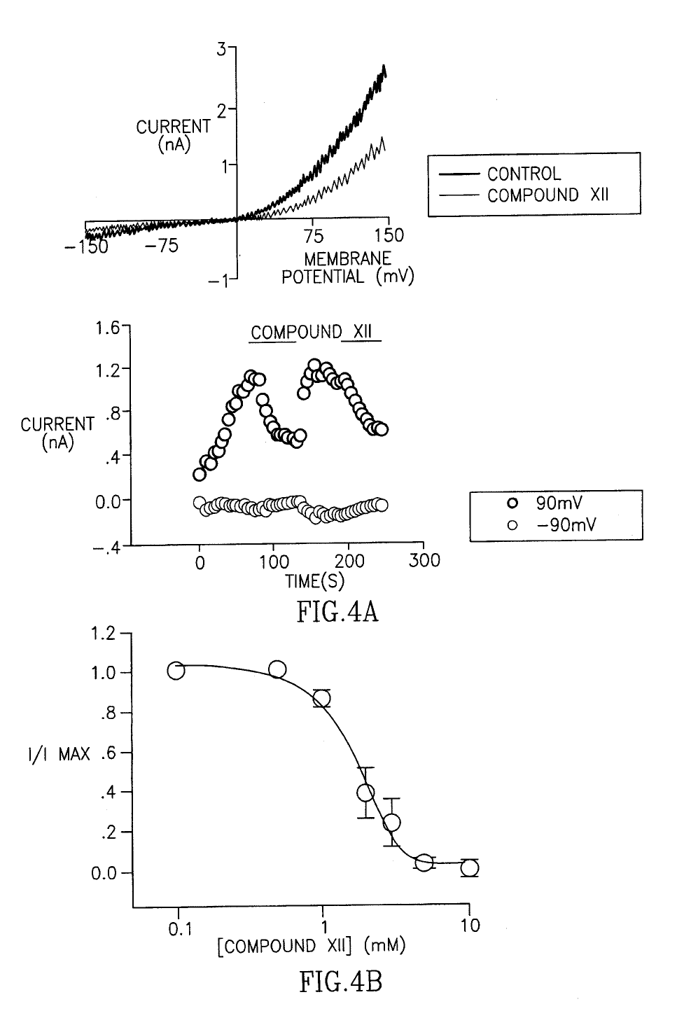

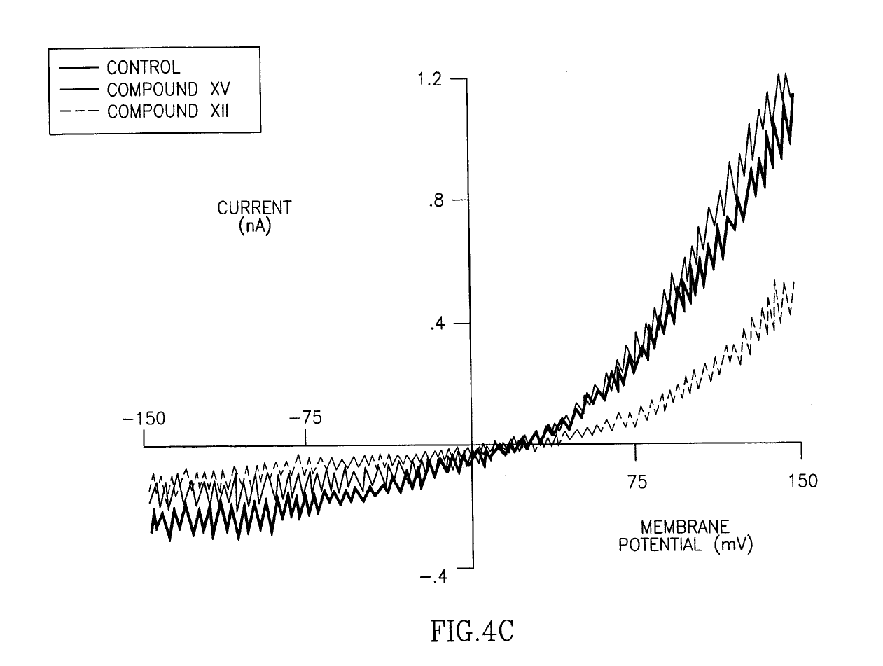

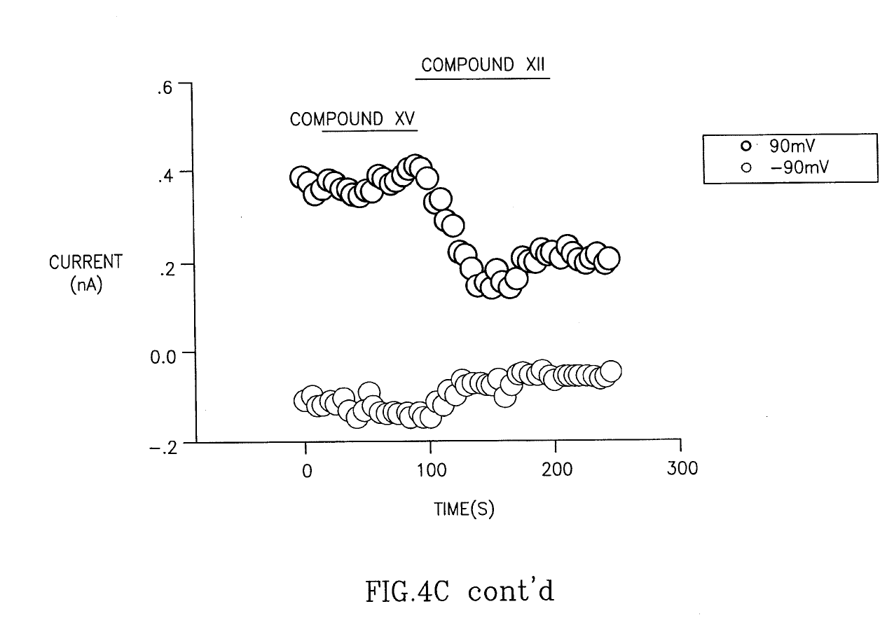

The phenol moiety is a common structure of Compound IX, Compound XIII and Compound XIV. To test the importance of the phenol moiety for inhibition of TRPL, Compound XII, a known thermo-TRP modulator that lacks a phenol moiety (Table 1), was tested. Compound XII inhibited TRPL activity (FIG. 4A) with an IC₅₀ of 1.6±0.3 mM (FIG. 4B). Inhibition of the TRPL channel by Compound XII was reversible, in contrast to TRPA1, which is activated by Compound XII channel irreversibly by covalent modification. Compound XV, a diphenol carboxylic acid that is otherwise similar to Compound XII, did not block TRPL at concentrations up to 5 mM (FIG. 4C); this may be due to its larger polarity compared to the other alkyl phenols examined.

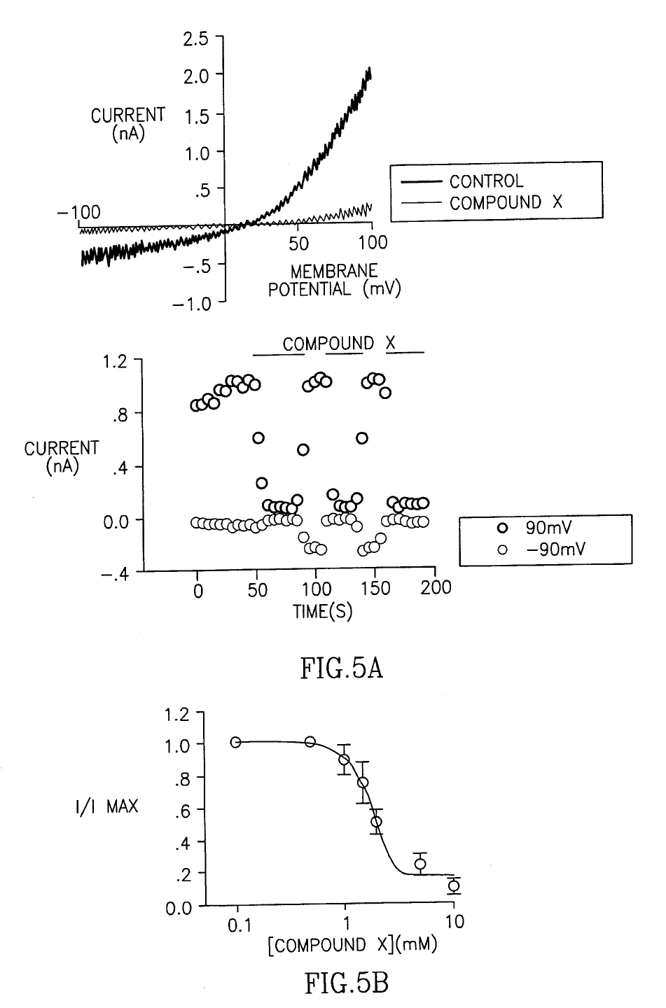

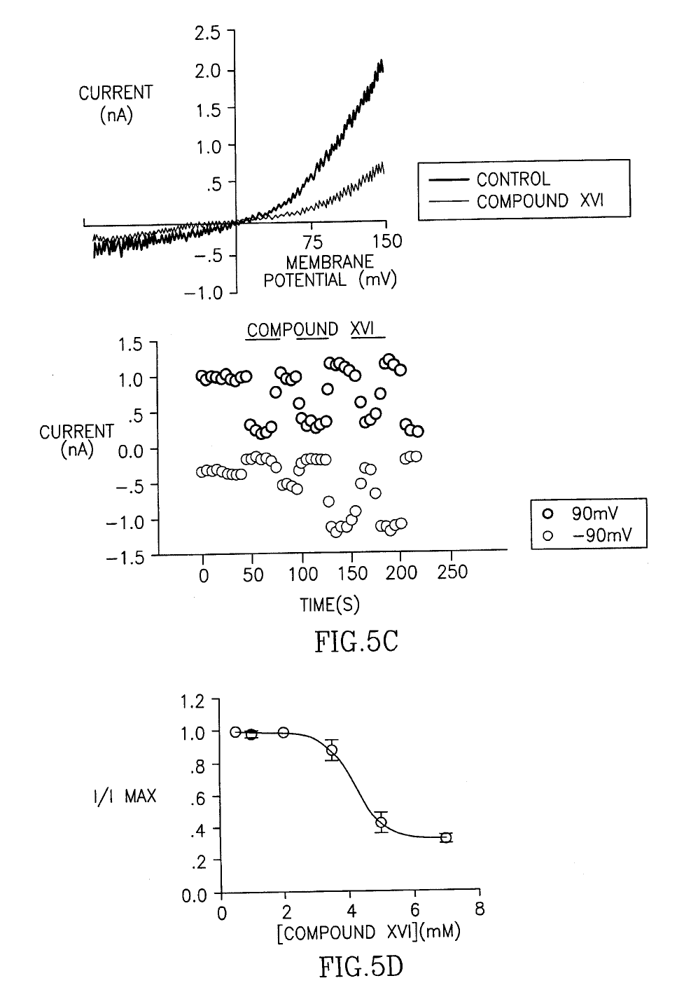

Thus, a phenol moiety is not necessary for inhibition of TRPL activity, as evidenced by the inhibitory activity of Compound XII, which lacks a phenol group. To test whether an aromatic moiety is required, Compound X and Compound XVI, which have a 6-hydrocarbon ring instead of an aromatic ring (Table 1), were tested. FIG. 5 shows that both Compound X and Compound XVI inhibited TRPL with IC₅₀'s of 1.81±0.13 mM and 4.22±0.4 mM, respectively, showing that the aromatic ring is not necessary for inhibition of TRPL. FIG. 5C shows an increase of inward current relative to outward current (i.e. a reduction of the outward rectification) with time. This phenomenon, which appeared occasionally for the different tested compounds, is not due to the development of a leak current, since it was always blocked completely by the tested compound. A similar phenomenon has been demonstrated in several types of mammalian TRP channels, and the underlying mechanism is still unknown.

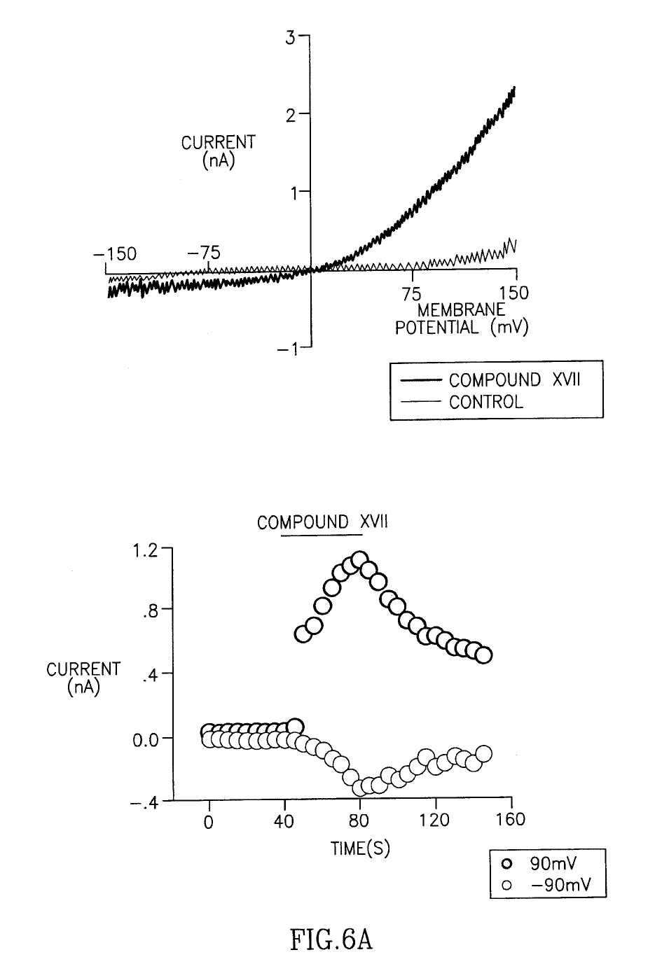

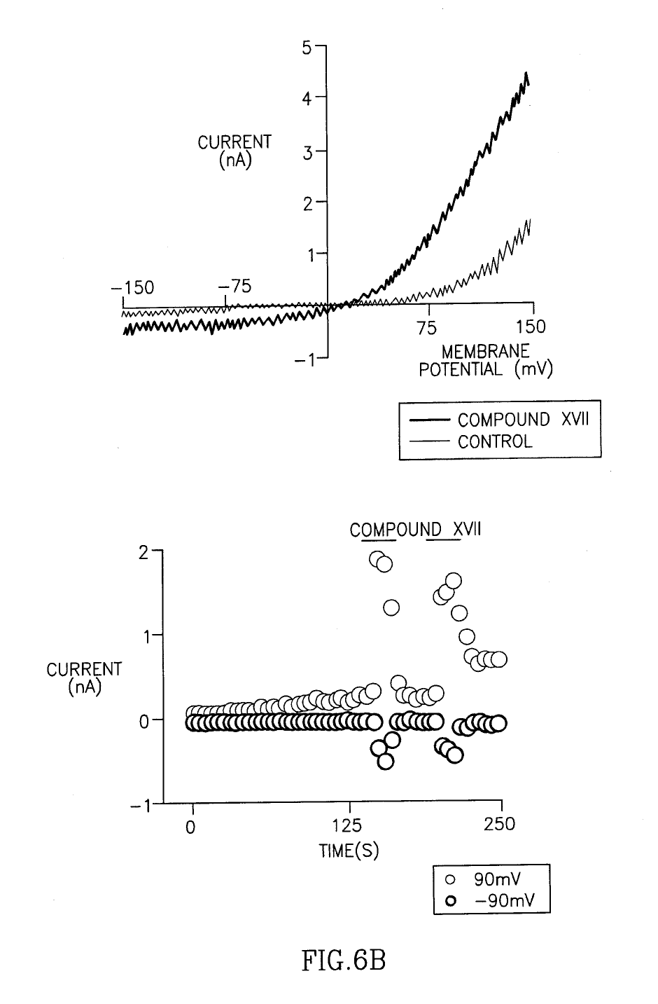

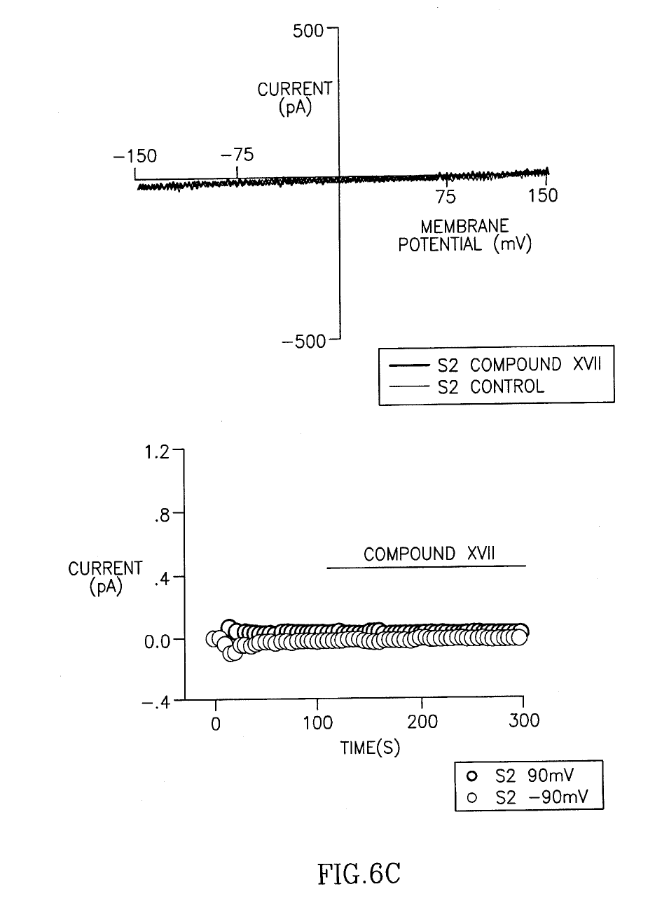

To examine whether other activators of TRPV3 that are not alkyl phenols also inhibit the TRPL channel, Compound XVII and Compound XI were tested. Interestingly, Compound XVII (5 mM FIG. 6A) and Compound XI (10 mM, Table 1) activated the TRPL channel. To make sure that the observed activation was not a manifestation of spontaneous increase in the basal activity of the channels, the basal constitutive current was allowed to increase to 2 nA, followed by addition of Compound XVII. FIG. 6B shows that a completely reversible rapid activation of the TRPL current was observed after addition of Compound XVII. Both enantiomers of Compound XVII were tested, and both were shown to have the same effect. Thus, these compounds unexpectedly activated TRPL.

### Example 3

### Compound IX Inhibits the Native TRPL Channels in Drosophila Photoreceptor Cells

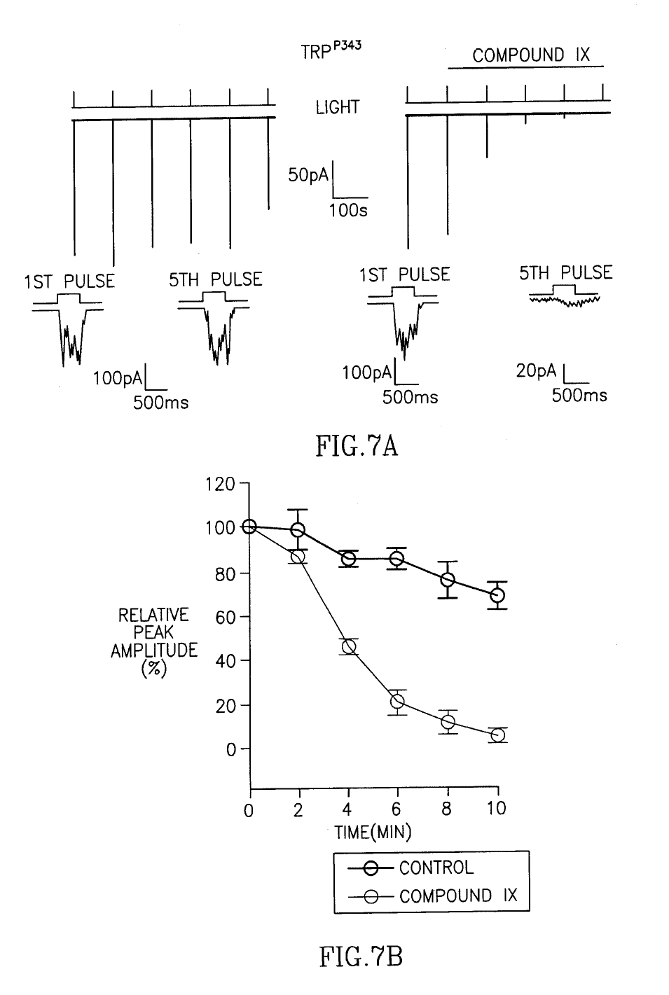

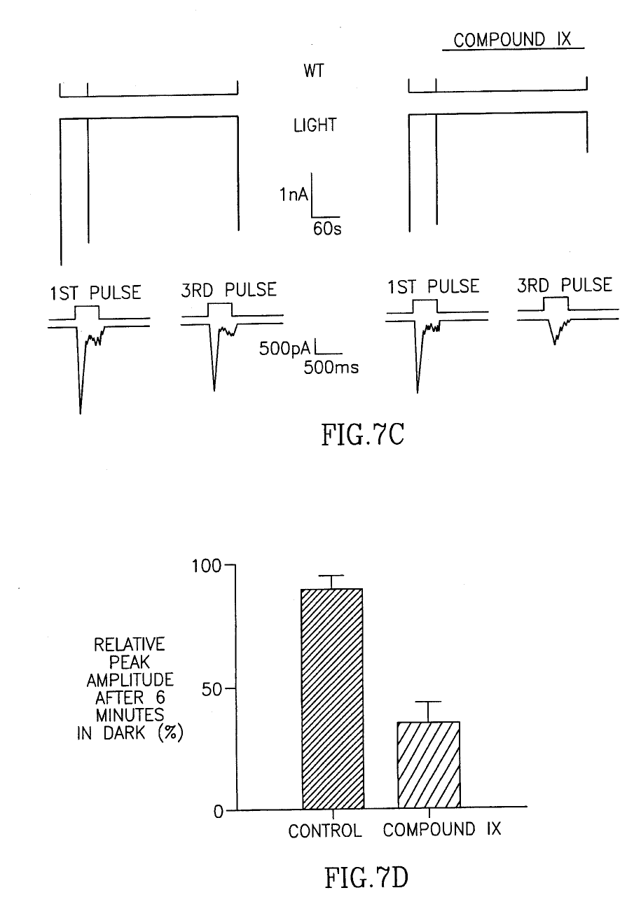

Although the biophysical properties of the expressed and native TRPL channels are similar, the effect of Compound IX on a typical TRPC channel in its native cells was tested to verify the data presented in the above Example. The effect of Compound IX on the native TRPL channel, one of the light-activated channels of Drosophila photoreceptor cells, was examined. This channel can be studied in isolation by measuring the light-induced current (LIC) in the Drosophila mutant trp^{P343}, which expresses only the TRPL channel. FIG. 7A, left panel shows LIC in response to a train of light pulses of constant intensity recorded from a photoreceptor cell of the trp^{P343} mutant. Application of Compound IX (500 μM) dramatically reduced LIC amplitude (FIG. 7A, right panel and FIG. 7B). In contrast to S2 cells expressing TRPL, in native photoreceptor cells the washout of lipophilic compounds is very slow (tens of min), and does not occur on a timescale in which the preparation remains viable. Inhibition of TRPL and TRP channels by 2-APB is enhanced by light stimulation (Chorna-Oman I et al, 2001). To examine whether light activation is required for the inhibitory effect of Compound IX on native TRPL channels, 2 light pulses were applied to the trp^{P343} mutant in the absence of inhibitor, then Compound IX was applied in the dark and the constant test light was applied again. This demonstrated that the inhibitory effect of Compound IX does not require light stimulation (FIG. 7C).

The use of Drosophila photoreceptor cells also enabled testing of the effect of Compound IX on the major light-activated channel TRP, which cannot be expressed in tissue culture cells. The light response of wild-type (WT) flies in response to intense lights is mediated mainly via TRP channels (Niemeyer B A et al, 1996); thus, WT flies were used to test the effect of Compound IX on TRP. Compound IX inhibited LIC of WT flies, demonstrating its inhibitor effect on both TRPL and TRP (FIGS. 7C-D).

The above experiments demonstrate that Compound IX inhibits TRPL channels, both native and ectopically expressed, and native TRP channels.

### Example 4

### Compound IX Inhibits Mammalian TRPM7 Expressed by HEK Cells

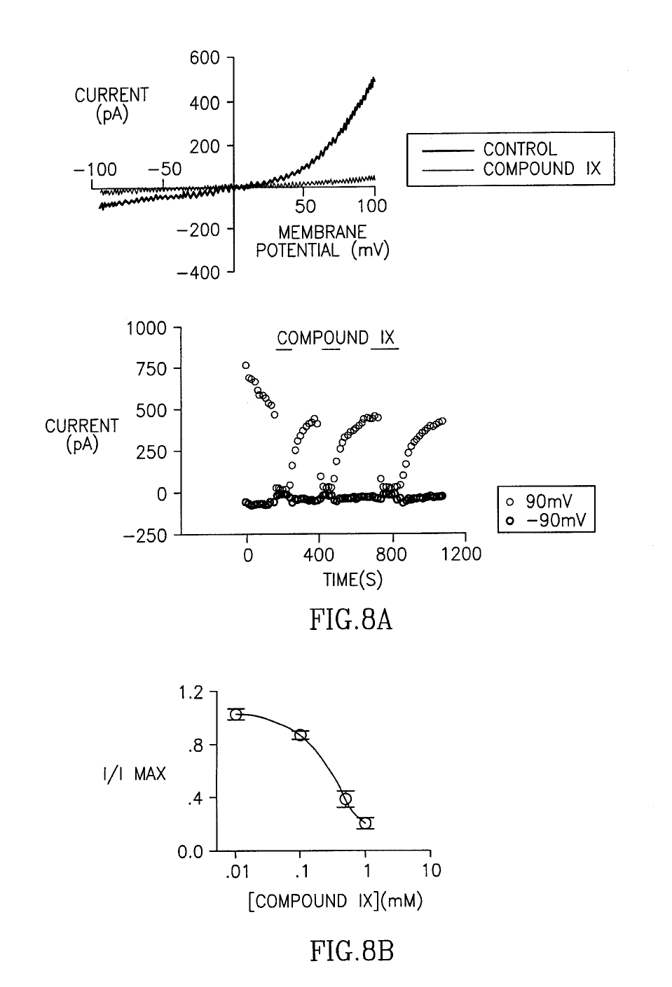

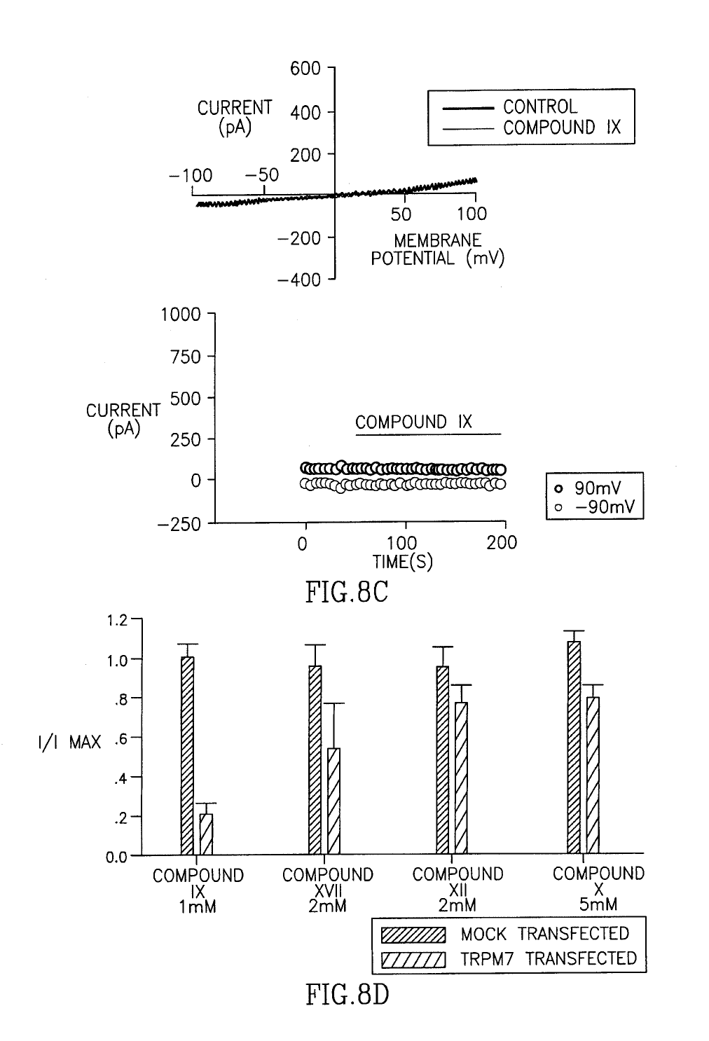

The TRPM7 channel, which belongs to the TRPM subfamily, has features similar to the TRPL channel, such as activation by anoxia and inhibition by divalent cations. Human Embryonic Kidney (HEK) cells were used to test the effect of Compound IX on TRPM7, since TRPM7 expressed in these cells is constitutively active in the absence of Mg-ATP. FIG. 8A, top panel depicts typical I-V curves of spontaneously active TRPM7 channels expressed in HEK293 cells, before (control) and after application of Compound IX. Before application of Compound IX, the normal outward rectifying I-V curve of the expressed TRPM7 channel can be seen. Large and dose-dependent suppression of the current was observed after application of Compound IX. Application of Compound IX reversibly blocked activity of the channels, as manifested by suppression of both inward and outward current (FIG. 8A), with an IC₅₀ of 306±65 μM (FIG. 8B), while no effect was observed in mock-transfected HEK293 cells (FIG. 8C). Compound XVII, Compound XII and Compound X were also found to inhibit TRPM7, although Compound IX had a more potent inhibitory effect than these 3 compounds (FIG. 8D).

### Example 5

### Compound IX Inhibits TRPM7 Ectopically Expressed in CA3-CA1 Hippocampal Cultures

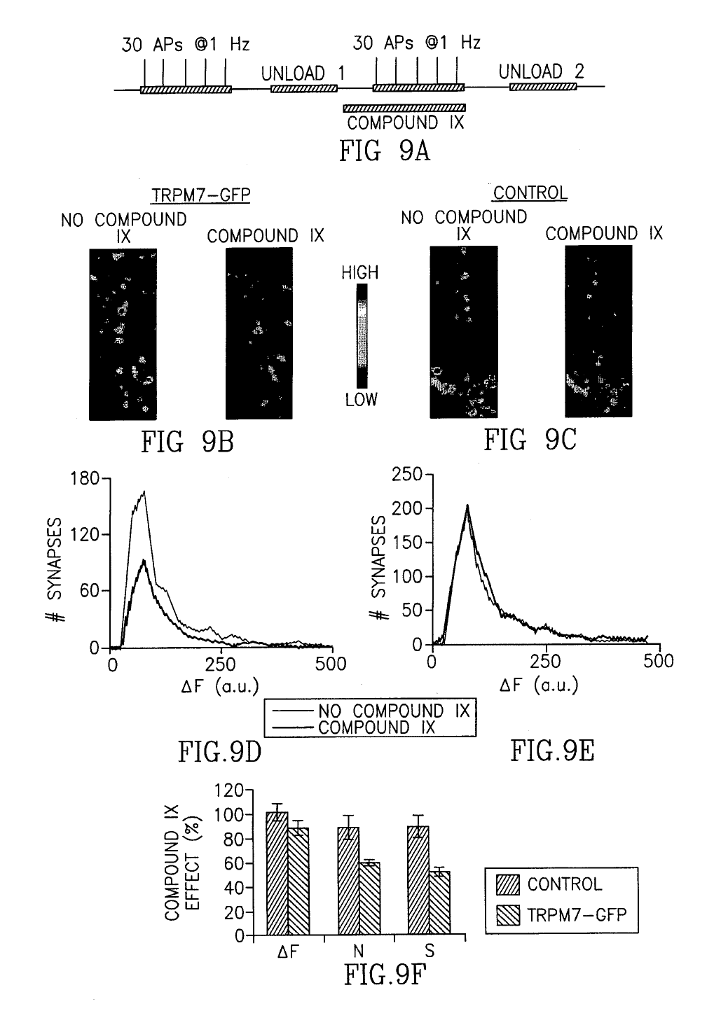

To test whether the inhibitory effect of Compound IX on TRPM7 is sufficient to block functional effects of the TRPM7 channel, the effect of Compound IX on TRPM7-dependent transmitter release was examined. Expressed TRPM7 channels enhance transmitter release in cholinergic sympathetic neurons. In rat hippocampal brain slices, activation of TRPM7 channels takes place only under oxidative stress conditions. Since over-expression of TRPM7 results in constitutively active channels, over-expression of TRPM7 in hippocampal brain neurons mimics stressful conditions in which active TRPM7 channels are involved. The effect of Compound IX was thus tested on the function of TRPM7 ectopically expressed in primary hippocampal CA1-CA3 neuron cultures. To test whether Compound IX affects synaptic vesicle release in hippocampal neurons, activity-dependent FM4-64 dye was used to directly monitor synaptic vesicle recycling. FIG. 9 compares vesicle release between control and TRPM7-GFP expressing synapses. Functional properties of synapses can be measured by estimating the probability of transmitter release and spatial distribution of functional presynaptic terminals. Thus, the number of vesicles released by a fixed number of simple spikes was measured (30 action potentials (APs) at 1 Hz, FIG. 9A). Fluorescent intensity of individual puncta (ΔF) reflects the number of vesicles released, whereas the number of fluorescent boutons reflects the number of active terminals that released at least one vesicle (N, corresponding to synapses with release probability>0.04). Total presynaptic strength (S) is estimated as the product of these two parameters (S=ΔF×N).

The effect of Compound IX on TRPM7-GFP expressing cells and on control cells not expressing TRPM7-GFP is shown in FIGS. 9B-C. Application of Compound IX (500 μM) significantly decreased vesicle release in TRPM7-GFP-expressing neurons (FIGS. 9D,F) but did not alter vesicle release in control neurons (FIGS. 9E-F). Reduction in vesicle release by TRPM7-GFP-expressing neurons was due to a decreased number of active terminals. There was no change in the average number of vesicles released in each active terminal (FIG. 9D). Compound IX inhibited the total presynaptic strength (S) by 47±3% but did not affect presynaptic activity in control neurons (FIG. 9F). Thus, Compound IX inhibits the function of mammalian TRPM7 expressed in synaptic terminals of hippocampal neurons.

In conclusion, Compound IX was shown in this and the above Example to inhibit TRPM7 expressed in both cell types tested, namely HEK cells and CA3-CA1 hippocampal primary cultured cells. Overall, the results presented above demonstrate that Compound IX inhibits channel members of the TRPC and TRPM subfamilies.

### Example 6

### Compound IX is Neuroprotective in a Mouse Model of Closed Head Injury

The neuroprotective effects of compounds of the present invention were next tested directly by testing their effect on recovery from closed head injury. NSS was determined at 1 h and 1, 2, 4, 5, 6, 7, 13, 20, and 27 days. While the mice did not recover completely due to the severity of the injury, NSS scores dropped significantly more in Compound IX-treated mice compared to negative control mice given saline injection (FIG. 10A); the difference in NSS between the 2 groups is depicted in FIG. 10B. Thus, compounds of the present invention exhibit a neuroprotective effect against brain damage.

### REFERENCES

Aarts M, lihara K, Wei W L et al. A key role for TRPM7 channels in anoxic neuronal death. Cell 2003; 115: 863-877.
Aarts M M, Tymianski M. TRPM7 and ischemic CNS injury. Neuroscientist 2005; 11: 116-123.
Agam K, von-Campenhausen M, Levy S et al. Metabolic stress reversibly activates the Drosophila light-sensitive channels TRP and TRPL in vivo. J Neurosci 2000; 20: 5748-5755.
Beck et al, TRPC channels determine human keratinocyte differentiation: new insight into basal cell carcinoma. Cell Calcium. 2008 May; 43(5):492-505.
Beni-Adani L et al, A peptide derived from activity-dependent neuroprotective protein (ADNP) ameliorates injury response in closed head injury in mice. J. Pharmacol. Exp. Ther. 296, 59-63, 2001.
Chen Y et al, An experimental model of closed head injury in mice: pathophysiology, histopathology, and cognitive deficits. J. Neurotrauma 13: 557-568, 1996.
Chorna-Oman I, Joel-Almagor T, Cohen-Ben A H et al. A common mechanism underlies vertebrate calcium signaling and Drosophila phototransduction. J Neurosci 2001; 21: 2622-2629.
Hu H Z, Gu Q, Wang C et al. 2-Aminoethoxydiphenyl borate is a common activator of TRPV1, TRPV2, and TRPV3. J Biol Chem 2004; 279: 35741-35748.
Lee S P, Buber M T, Yang Q et al. Thymol and related alkyl phenols activate the hTRPA1 channel. Br J Pharmacol 2008; 153: 1739-1749.
Macpherson L J, Hwang S W, Miyamoto T, Dubin A E, Patapoutian A, Story G M. More than cool: promiscuous relationships of menthol and other sensory compounds. Mol Cell Neurosci 2006; 32: 335-343.
Minke B, Cook B. TRP channel proteins and signal transduction. Physiol Rev 2002; 82: 429-472.
Minke B, Wu C, Pak W L. Induction of photoreceptor voltage noise in the dark in Drosophila mutant. Nature 1975; 258: 84-87.
Moqrich A, Hwang S W, Earley T J et al. Impaired thermosensation in mice lacking TRPV3, a heat and camphor sensor in the skin. Science 2005; 307: 1468-1472.
Niemeyer B A, Suzuki E, Scott K, Jalink K, Zuker C S. The Drosophila light-activated conductance is composed of the two channels TRP and TRPL. Cell 1996; 85: 651-659.
Nilius et al, Transient receptor potential cation channels in disease. Physiol Rev. 2007; 87(1):165-217.
Parnas M, Katz B, Minke B. Open channel block by Ca2+ underlies the voltage dependence of Drosophila TRPL channel. J Gen Physiol 2007; 129: 17-28.
Peretz A, Suss-Toby E, Rom-Glas A, Anion A, Payne R, Minke B. The light response of Drosophila photoreceptors is accompanied by an increase in cellular calcium: effects of specific mutations. Neuron 1994; 12: 1257-1267.
Slutsky I, Sadeghpour S, Li B, Liu G. Enhancement of synaptic plasticity through chronically reduced Ca2+ flux during uncorrelated activity. Neuron 2004; 44: 835-849.
Stokes et al, A TRPV2-PKA signaling module for transduction of physical stimuli in mast cells, J Exp Med. 2004 Jul. 19; 200(2):137-47.
Vogt-Eisele A K, Weber K, Sherkheli M A et al. Monoterpenoid agonists of TRPV3. Br J Pharmacol 2007; 151: 530-540.
Xu H, Delling M, Jun J C, Clapham D E. Oregano, thyme and clove-derived flavors and skin sensitizers activate specific TRP channels. Nat Neurosci 2006; 9: 628-635.

1. A composition comprising a compound of Formula I: 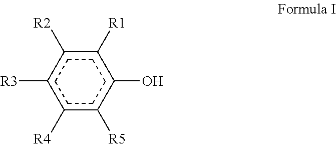
or a hydrous or anhydrous salt or metastable or stable polymorph thereof,
wherein the structure 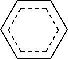
refers to a 6-carbon ring that is either aromatic or has 0, 1, or 2 double bonds;
wherein R¹, R², R³, R⁴, and R⁵ are independently selected from the group consisting of:
H;
C₁₋₄ alkyl that is unsubstituted or substituted with 1-4 substituents, independently selected from the group consisting of I; Br; Cl; F; nitro; amino; —OH; ═O; C₁-C₃ alkoxy; C₁-C₃ thioalkoxy; C₁-C₃ haloalkyl with 1 to 6 halogen substitutions; C₁-C₃ haloalkoxy with 1 to 6 halogen substitutions; and C₁-C₃ heteroalkyl with 1 to 2 heteroatoms selected from the group consisting of O, N and S;
C₁₋₄ alkoxy that is unsubstituted or substituted with 1-4 substituents as recited above for C₁₋₄ alkyl;
CF₃; OH; I; Br; Cl; F; NH₃; and NO₂;
with the proviso that at least one of R¹, R², R³, R⁴, and R⁵ is C₁₋₄ alkyl that is unsubstituted or substituted as recited above,
for treating or inhibiting neuronal damage.

2. The composition of claim 1, wherein the structure of said compound is set forth in Formula II: 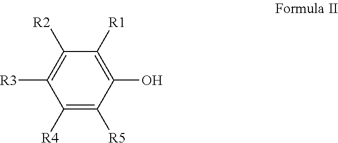
wherein all components are as defined for claim 1.

3. The composition of claim 1, wherein the structure of said compound is set forth in Formula III: 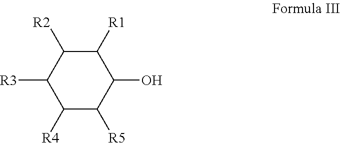
wherein all components are as defined for claim 1.

4. The composition of any of claims 1-3, wherein at least of R¹, R², R³, R⁴, and R⁵ is a C₁₋₄ alkyl moiety selected from the group consisting of an isopropenol moiety, an isopropenyl moiety and a tert-butyl moiety, wherein said moiety is unsubstituted or is substituted with 1-4 substituents, independently selected from the group consisting of I; Br; Cl; F; nitro; amino; —OH; ═O; C₁-C₃ alkoxy; C₁-C₃ thioalkoxy; C₁-C₃ haloalkyl with 1 to 6 halogen substitutions; C₁-C₃ haloalkoxy with 1 to 6 halogen substitutions; and C₁-C₃ heteroalkyl with 1 to 2 heteroatoms selected from the group consisting of O, N and S.

5. The composition of claim 4, wherein said isopropenol moiety, isopropenyl moiety or tert-Butyl moiety is unsubstituted.

6. The composition of claim 4, wherein, in addition to said isopropenol moiety, isopropenyl moiety or tert-Butyl moiety, at least one of R¹, R², R³, R⁴, and R⁵ is C₁₋₄ alkyl that is unsubstituted or substituted with 1-4 substituents, independently selected from the group consisting of I; Br; Cl; F; nitro; amino; —OH; ═O; C₁-C₃ alkoxy; C₁-C₃ thioalkoxy; C₁-C₃ haloalkyl with 1 to 6 halogen substitutions; C₁-C₃ haloalkoxy with 1 to 6 halogen substitutions; and C₁-C₃ heteroalkyl with 1 to 2 heteroatoms selected from the group consisting of O, N and S.

7. The composition of any of claims 1-6, wherein said neuronal damage is acute.

8. A composition comprising a compound of Formula VIII 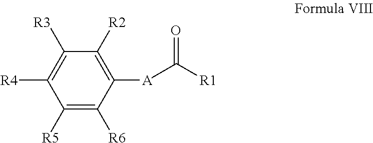
or a hydrous or anhydrous salt or metastable or stable polymorph thereof,
wherein R¹ is selected from the group consisting of H and —OH;
A is C₁₋₄ alkyl that is unsubstituted or substituted with 1-4 substituents, wherein said substituents are independently selected from the group consisting of I; Br; Cl; F; nitro; amino; —OH; ═O; C₁-C₃ alkoxy; C₁-C₃ thioalkoxy; C₁-C₃ haloalkyl with 1 to 6 halogen substitutions; C₁-C₃ haloalkoxy with 1 to 6 halogen substitutions; and C₁-C₃ heteroalkyl with 1 to 2 heteroatoms selected from the group consisting of O, N and S; and
R², R³, R⁴, R⁵, and R⁶ are independently selected from the group consisting of:
H;
C₁₋₄ alkyl which is unsubstituted or substituted with 1-4 substituents, wherein said substituents are independently selected from the group consisting of I; Br; Cl; F; nitro; amino; —OH; ═O; C₁-C₃ alkyl; C₁-C₃ alkoxy; C₁-C₃ thioalkoxy; C₁-C₃ haloalkyl with 1 to 5 halogen substitutions; C₁-C₃ haloalkoxy with 1 to 5 halogen substitutions; and C₁-C₃ heteroalkyl with 1 to 3 heteroatoms selected from the group consisting of O, N and S;
C₁₋₄ alkoxy that is unsubstituted or substituted with 1-4 substituents as recited above for C₁₋₄ alkyl;
CF₃; OH; I; Br; Cl; F; NH₃; and NO₂;
for treating or inhibiting neuronal damage.

9. The composition of claim 8, wherein R², R³, R⁴, R⁵, and R⁶ are all H.

10. The composition of claim 8, wherein A is C₁₋₃ alkyl which is unsubstituted.

11. The composition of claim 8, wherein AC═OR¹ has a structure as represented in Formula VII:

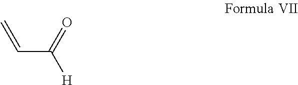

12. The composition of claim 8, wherein said neuronal damage is acute.

13. A composition comprising a compound of Formula IV: 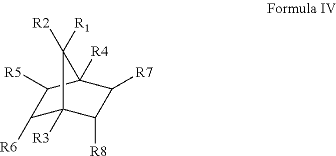
or a hydrous or anhydrous salt or metastable or stable polymorph thereof,
wherein R¹, R², R³, and R⁴ are independently selected from the group consisting of methyl which is unsubstituted or substituted with 1-3 substituents, wherein said substituents are independently selected from the group consisting of I; Br; Cl; F; nitro; amino; —OH; and ═O;
wherein R⁵, R⁶, and R⁷ are independently selected from the group consisting of:
H;
C₁₋₂ alkyl which is unsubstituted or substituted with 1-3 substituents, wherein said substituents are independently selected from the group consisting of I; Br; Cl; F; nitro; amino; —OH; and ═O;
C₁₋₂ alkoxy that is unsubstituted or substituted with 1-4 substituents as recited above for C₁₋₄ alkyl;
CF₃; OH; I; Br; Cl; F; NH₃; and NO₂; and
wherein R⁸ is selected from the group consisting of —OH and ═O,
for treating pruritus secondary to an inflammatory disorder selected from the group consisting of psoriasis, eczema, sunburn, allergic contact dermatitis (ACD), and physical urticaria; treating a disease selected from the group consisting of Darier's and Hailey Hailey's diseases; or reducing the incidence of basal cell carcinoma (BCC).

14. The composition of claim 13, wherein R¹, R², R³, and R⁴ are all unsubstituted methyl.

15. The composition of claim 13, wherein R⁵, R⁶, and R⁷ are all H.

16. A pharmaceutical composition for treating or inhibiting neuronal damage, comprising a pharmaceutically acceptable carrier and as an active ingredient the compound of any one of claims 1 to 12.

17. Use of the compound of any one of claims 1 to 12 for the preparation of a medicament for the treating or inhibiting neuronal damage.

18. A method for treating or inhibiting neuronal damage in a patient, said method comprising the step of administering to the patient a therapeutically effective amount of the compound of any one of claims 1 to 12.

19. A method for inhibiting signaling by a TRPC (Canonical Transient Receptor Potential) channel of a patient, said method comprising the step of administering to the patient a therapeutically effective amount of the compound of any one of claims 1 to 12.

20. A method for inhibiting signaling by a non-thermo-Melastatin-subfamily Transient Receptor Potential (TRPM) channel, said method comprising the step of administering to a subject in need of such treatment a therapeutically effective amount of the compound of any one of claims 1 to 12.

21. A method for treating pruritus secondary to an inflammatory disorder selected from the group consisting of psoriasis, eczema, sunburn, allergic contact dermatitis (ACD), and physical urticaria; treating a disease selected from the group consisting of Darier's and Hailey Hailey's diseases; or reducing the incidence of basal cell carcinoma (BCC), said method comprising the step of administering to the patient a therapeutically effective amount of the compound of any one of claims 13 to 15.

22. A method for activating signaling by a TRPC (Canonical Transient Receptor Potential) channel of a patient, said method comprising the step of administering to the patient a therapeutically effective amount of the compound of any one of claims 13 to 15.

23. A method for activating signaling by a non-thermo-Melastatin-subfamily Transient Receptor Potential (TRPM) channel, said method comprising the step of administering to a subject in need of such treatment a therapeutically effective amount of the compound of any one of claims 13 to 15.
